(19) United States
(12) Patent Application Publication
(10) Pub. No.: US 2025/0108049 A1
(43) Pub. Date: Apr. 3, 2025

(54) COMPOUNDS FOR TREATING MDS-ASSOCIATED ANEMIAS AND OTHER CONDITIONS

(71) Applicant: Agios Pharmaceuticals, Inc., Cambridge, MA (US)

(72) Inventors: Vanessa Beynon, Cambridge, MA (US); Melissa L. DiBacco, Glastonbury, CT (US); Rolandas Urbstonaitis, Revere, MA (US); Megan Lynch, Concord, MA (US); Suman Joy Bhatia, Newtown, PA (US); Lenny Dang, Boston, MA (US); Varsha V. Iyer, Boston, MA (US); Chi-yun Charles Kung, Arlington, MA (US); Zhen Xiao, Arlington, MA (US); Ophelia Qiping Yin, Westfield, NJ (US)

(21) Appl. No.: 18/711,090

(22) PCT Filed: Nov. 15, 2022
(86) PCT No.: PCT/US2022/049969

§ 371 Date: May 16, 2024

Related U.S. Application Data
(60) Provisional application No. 63/280,069, filed on Nov. 16, 2021.
(60) Provisional application No. 63/357,240, filed on Jun. 30, 2022.

(51) Int. Cl. A61K 31/5025; A61P 7/06

(52) U.S. Cl. CPC A61K 31/5025; A61P 7/06

(57) ABSTRACT

Provided herein is the use of certain pyruvate kinase activators or pharmaceutically acceptable salts or compositions thereof for treating MDS-associated anemia and other conditions.

### RELATED APPLICATIONS

This application claims priority to U.S. Provisional Application No. 63/280,069, filed Nov. 16, 2021 and U.S. Provisional Application No. 63/357,240, filed Jun. 30, 2022, each of which are incorporated herein in their entirety.

### BACKGROUND

Myelodysplastic syndromes (MDS) are a heterogeneous group of rare hematological malignancies characterized by dysfunctional hematopoiesis, progressive cytopenia, and an increased risk of progression to acute myeloid leukemia (AML). MDS occurs when the blood-forming cells in the bone marrow become abnormal (dysplastic) and have problems making new blood cells. Many of the blood cells formed by the bone marrow cells often die or are destroyed by the body, thus leaving the individual without enough normal blood cells. Although different cell types are affected by this phenomenon, the most common finding in MDS is a shortage of red blood cells (anemia). However, hemolytic anemia has also been found to occur, albeit rarely, in patients suffering from MDS. See, for example, Leukemia Research Reports, Vol. 5, 2016, pp 23-26.

With an average age at diagnosis of 71 years, MDS patients can be severely impacted by chronically low levels of hemoglobin. This can lead to fatigue, worsening cardiopulmonary function, increased falls, and significant cognitive decline. As such, treatment of anemia is essential for overall health and quality of life. Currently, transfusions of packed red blood cells (PRBC) is the standard of care for MDS-associated anemia. The problem with transfusion dependent MDS-associated anemia patients, however, is that these patients are at a higher risk of iron overload and transfusion reactions and report a decreased quality of life.

Alternative approaches for treating MDS-associated anemias are therefore needed.

### SUMMARY

It has now been found that certain activators of pyruvate kinase may also be effective in treating MDS-associated anemia, particularly anemia associated with very low-risk, low-risk and intermediate-risk MDS. These activators include those having the following structure formulae:

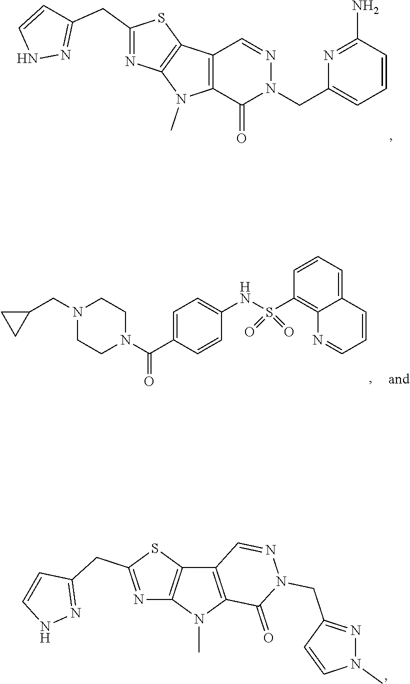 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

as well as pharmaceutically acceptable salts thereof. In one aspect, the disclosed pyruvate kinase activators have been found to increase hemoglobin levels, decrease reticulocyte count, and/or improve red blood cell (RBC) functionality in animal models of MDS. See, e.g., FIGS. 1 and 2.

Provided herein, therefore, are methods of treating MDS-associated anemias and other conditions in a subject, comprising administering to the subject one or more of the disclosed pyruvate kinase activators, or a pharmaceutically acceptable salt or composition thereof.

Also provided are methods for increasing the hemoglobin level in a subject suffering from MDS, comprising administering to the subject one or more of the disclosed pyruvate kinase activators, or a pharmaceutically acceptable salt or composition thereof.

Also provided are methods of treating acquired PK deficiency (PKD) in a subject suffering from MDS, comprising administering to the subject one or more of the disclosed pyruvate kinase activators, or a pharmaceutically acceptable salt or composition thereof.

Also provided are methods of treating anemias associated with acquired PKD in a subject suffering from MDS, comprising administering to the subject one or more of the disclosed pyruvate kinase activators, or a pharmaceutically acceptable salt or composition thereof.

Also provided are methods of treating cytopenia in a subject suffering from MDS, comprising administering to the subject one or more of the disclosed pyruvate kinase activators, or a pharmaceutically acceptable salt or composition thereof.

Further provided are methods of treating hemolytic anemia in a subject suffering from MDS, comprising administering to the subject one or more of the disclosed pyruvate kinase activators, or a pharmaceutically acceptable salt or composition thereof.

Further provided are methods of treating ineffective erythropoiesis in a subject suffering from MDS, comprising administering to the subject one or more of the disclosed pyruvate kinase activators, or a pharmaceutically acceptable salt or composition thereof.

Further provided are methods of reducing disease progression in subjects suffering from MDS. In some aspects, administering to the subject one or more of the disclosed pyruvate kinase activators, or a pharmaceutically acceptable salt or composition thereof reduces inflammation within the subject's bone marrow thereby reducing disease progression in the subject.

Still further provided are methods of treating diseases related to mitochondrial dysfunction in a subject in need thereof, comprising administering to the subject one or more of the disclosed pyruvate kinase activators, or a pharmaceutically acceptable salt or composition thereof.

### BRIEF DESCRIPTION OF THE DRAWINGS

FIGS. 1A-D show the hemoglobin level effect in an MDS-associated anemia mouse model following administration of mitapivat in chow for 6 weeks (FIG. 1A), 8 weeks (FIG. 1B), 12 weeks (FIG. 1C), and 18 weeks (FIG. 1D).

FIGS. 2A-D show hematological parameter results for red blood cell count (RBC) (FIG. 2A), reticulocyte percentage (FIG. 2B), reticulocyte concentration (FIG. 2C), and hematocrit percentage (FIG. 2D) from an MDS-associated anemia mouse model following administration of mitapivat.

FIG. 3 shows a multi-phase clinical trial design for the treatment of MDS-associated anemia.

FIG. 4 shows an overview of a Phase 2a study design with Compound 1 in subjects with anemia due to lower-risk myelodysplastic syndromes (LR-MDS).

FIG. 5 shows an overview of a Phase 2b study design with Compound 1 in subjects with anemia due to lower-risk myelodysplastic syndromes (LR-MDS).

FIGS. 6A-E show flow cytometry data of bone marrow aspirates from Polg^{D257A} mice following treatment with Compound 1.

FIGS. 7A-D show flow cytometry data of bone marrow aspirates from NHD13 mice following treatment with mitapivat or Compound 1.

FIGS. 8A-C show hematological parameter results for hemoglobin (FIG. 8A), RBC (FIG. 8B), and reticulocyte concentration (FIG. 8C) from NHD13 mice following treatment with mitapivat or Compound 1.

### DETAILED DESCRIPTION

In a first embodiment, provided herein is a method for treating anemia associated with myelodysplastic syndrome (MDS) in a subject suffering from MDS comprising administering to the subject a therapeutically effective amount of a compound having a structural formula selected from:

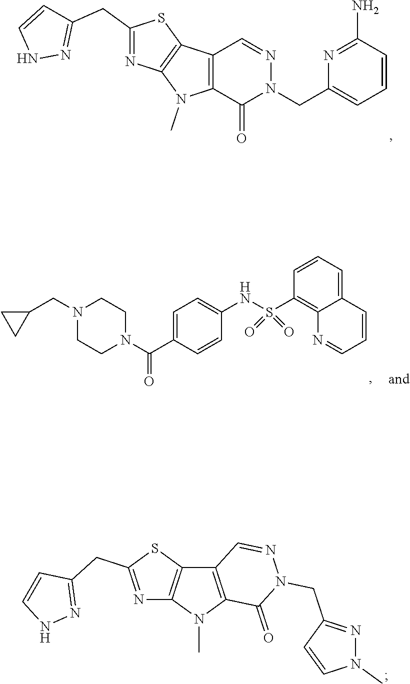 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

In a second embodiment, provided herein is a method for treating hemolytic anemia associated with myelodysplastic syndrome (MDS) in a subject suffering from MDS comprising administering to the subject a therapeutically effective amount of a compound having a structural formula selected from:

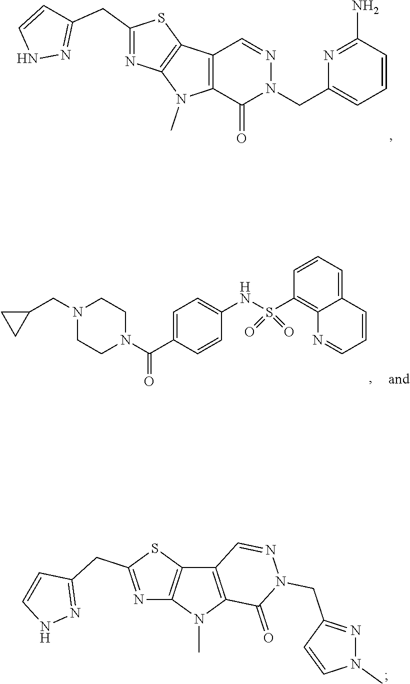 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

In a third embodiment, provided herein is a method of increasing the hemoglobin level in a subject suffering from myelodysplastic syndrome (MDS) comprising administering to the subject a therapeutically effective amount of a compound having a structural formula selected from:

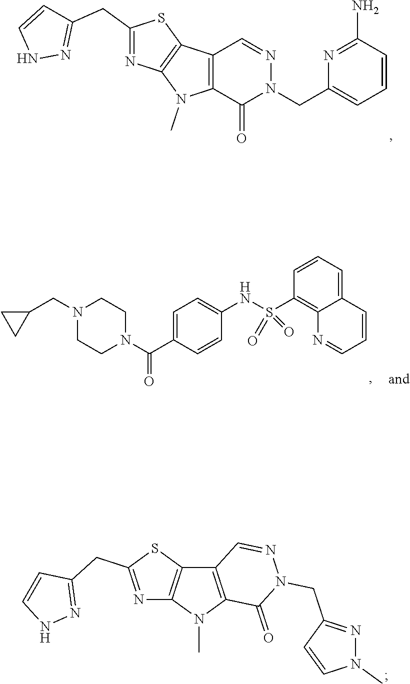 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

In a fourth embodiment, provided herein is a method of treating acquired PK deficiency (PKD) in a subject suffering from myelodysplastic syndrome (MDS) comprising administering to the subject a therapeutically effective amount of a compound having a structural formula selected from:

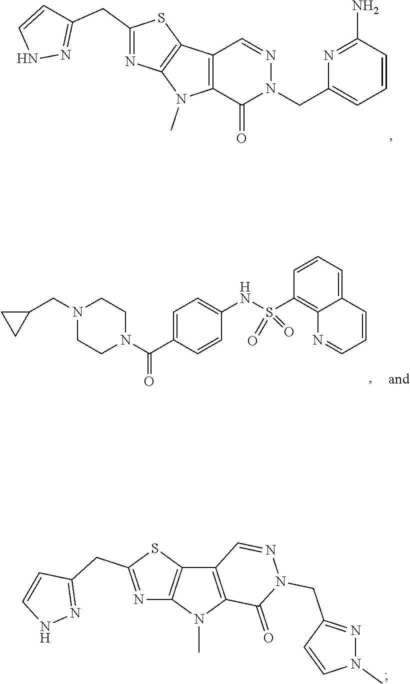 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

In a fifth embodiment, provided herein is a method of treating anemia associated with acquired PK deficiency (PKD) in a subject suffering from myelodysplastic syndrome (MDS) comprising administering to the subject a therapeutically effective amount of a compound having a structural formula selected from:

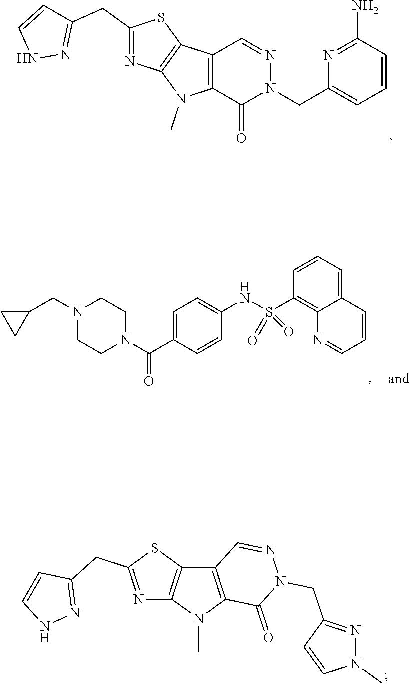 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

In a sixth embodiment, provided herein is a method of treating cytopenia in a subject suffering from myelodysplastic syndrome (MDS) comprising administering to the subject a therapeutically effective amount of a compound having a structural formula selected from:

or a pharmaceutically acceptable salt of any of the foregoing.

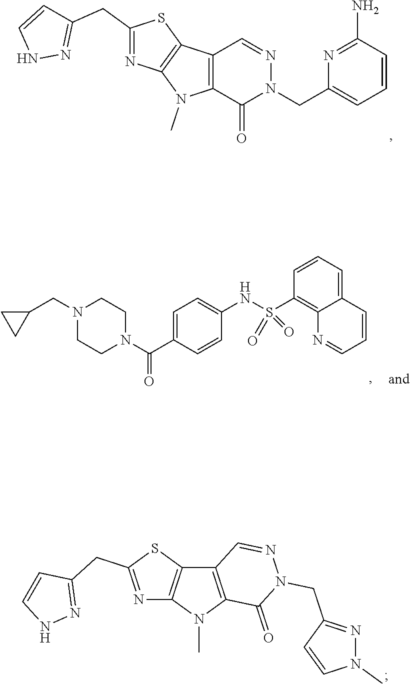 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

In a seventh embodiment, provided herein is a method of treating diseases related to mitochondrial dysfunction in a subject in need thereof comprising administering to the subject a therapeutically effective amount of a compound having a structural formula selected from:

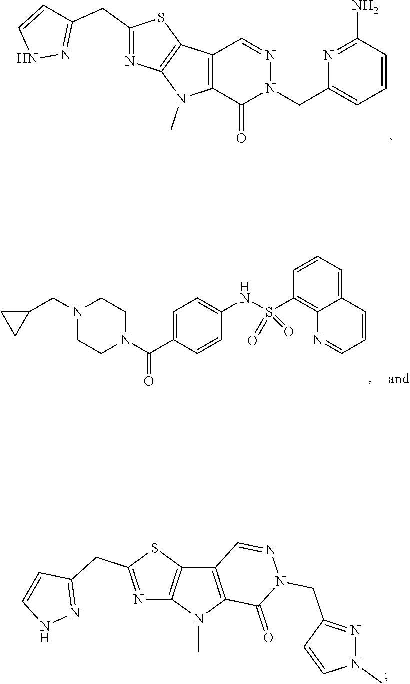 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

In an eighth embodiment, provided herein is a method of treating ineffective erythropoiesis in a subject in need thereof comprising administering to the subject a therapeutically effective amount of a compound having a structural formula selected from:

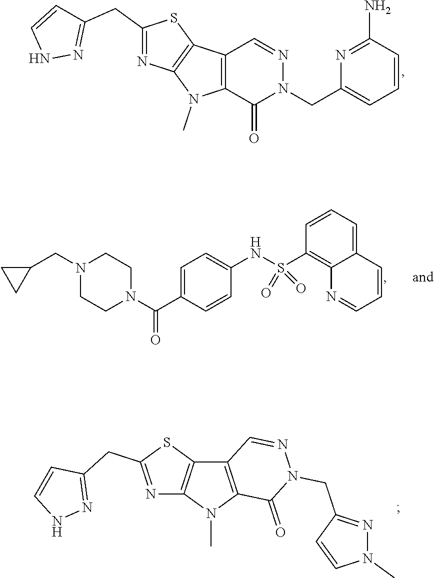 [SMILES: Cn1c2nc(Cc3cc[nH]n3)sc2c2cnn(Cc3cccc(N)n3)c(=O)c21 ; O=C(c1ccc(NS(=O)(=O)c2cccc3cccnc23)cc1)N1CCN(CC2CC2)CC1 ; Cn1ccc(Cn2ncc3c4sc(Cc5cc[nH]n5)nc4n(C)c3c2=O)n1]

or a pharmaceutically acceptable salt of any of the foregoing.

As used herein the term “hemolytic anemia” refers to a sub-type of anemia where a subject's low red blood cell count is caused by the destruction rather than the underproduction of red blood cells. Unless stated otherwise the term “anemia” as used herein refers to a low red blood cell count that is caused by underproduction of red blood cells, including ineffective erythropoiesis.

The terms “MDS-associated anemia”, “anemia associated with MDS”, and “anemia due to MDS” are synonymous and refer to anemia that has developed or has been acquired in a subject as a result of having or suffering from MDS.

The terms “anemia associated with acquired PK deficiency (PKD)” and “PKD-associated anemia” are synonymous and refer to anemia which has developed in a subject as a result of an acquired PKD that arises from having or suffering from MDS. In some embodiments, the anemia associated with acquired PK deficiency in a subject suffering from MDS is hemolytic anemia.

The terms “subject” and “patient” are synonymous and refer to a mammal in need of treatment, e.g., companion animals (e.g., dogs, cats, and the like), farm animals (e.g., cows, pigs, horses, sheep, goats, and the like) and laboratory animals (e.g., rats, mice, guinea pigs, and the like). Unless stated to the contrary, the subject is a human in need of treatment. In some embodiments the subject is an adult human (e.g.,≥18 years of age). In other embodiments the subject is a human child (e.g., <18 years of age). In still other embodiments the subject is a human female (adult or child). In yet other embodiments the subject is a human male (adult or child).

The terms “administer,” “administering,” or “administration” refer to providing, implanting, absorbing, ingesting, injecting, inhaling, or otherwise introducing a compound described herein, or a pharmaceutically acceptable salt or composition thereof, to, in or on a subject.

The terms “treatment,” “treat,” and “treating” refer to reversing, alleviating, delaying the onset of, or inhibiting the progress of a disease or one or more symptoms of a disease described herein. In some embodiments, treatment may be administered after one or more signs or symptoms of the disease have developed or have been observed (i.e., therapeutic treatment). In other embodiments, treatment may be administered in the absence of signs or symptoms of the disease. For example, treatment may be administered to a susceptible subject prior to the onset of symptoms (i.e., prophylactic treatment) (e.g., in light of a history of symptoms and/or in light of exposure to a pathogen). Treatment may also be continued after symptoms have resolved, for example, to delay or prevent recurrence. In certain embodiments, treatment includes delaying onset of at least one symptom of the disorder for a period of time.

The terms an “effective amount” or “therapeutically effective amount” of a compound or a pharmaceutically acceptable salt thereof described herein refer to an amount of a compound or a pharmaceutically acceptable salt thereof that is sufficient to provide a therapeutic benefit in the treatment of a condition described herein. In one aspect, an effective amount is between about 0.01 to about 100 mg/kg body weight/day of a provided compound or pharmaceutically acceptable salt, such as, e.g., about 0.1 to about 100 mg/kg body weight/day. In another aspect, an effective amount is between about 0.01 to about 2000 mg of a provided compound or a pharmaceutically acceptable salt thereof which may be administered once or twice daily.

As used herein, the recitation of a range of values is intended to serve as a shorthand method of referring individually to each separate value falling within the range as well as the highest and lowest values that define the range and each value is incorporated into the specification as if it were individually recited herein, unless expressly stated to the contrary. For example, a range of values from X to Y includes both X and Y and all the values in between X and Y.

The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art, for example, Berge et al. describe pharmaceutically acceptable salts in detail in J. Pharmaceutical Sciences, 1977, 66, 1-19. Pharmaceutically acceptable salts of the compounds disclosed herein include those derived from suitable inorganic and organic acids. Examples of pharmaceutically acceptable acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, besylate bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, gentisate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, tosylate p-toluenesulfonate, undecanoate, valerate salts, and the like.

In one aspect, as part of a ninth embodiment, the compound administered to the subject (e.g., as in any one of the first to eighth embodiments), is of the structural formula:

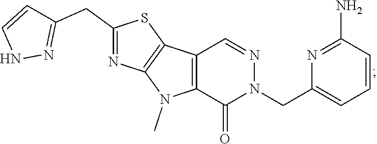 [SMILES: Cn1c2nc(Cc3cc[nH]n3)sc2c2cnn(Cc3cccc(N)n3)c(=O)c21]

or a pharmaceutically acceptable salt thereof. This compound is also referred to herein by its chemical name 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one, or simply as Compound 1.

In one aspect, as part of a tenth embodiment, the compound administered to the subject (e.g., as in any one of the first to eighth embodiments), is of the structural formula:

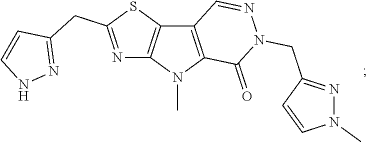 [SMILES: Cn1ccc(Cn2ncc3c4sc(Cc5cc[nH]n5)nc4n(C)c3c2=O)n1]

or a pharmaceutically acceptable salt thereof. This compound is also referred to herein by its chemical name 2-((1H-pyrazol-3-yl)methyl)-4-methyl-6-((1-methyl-1H-pyrazol-3-yl)methyl)-4H-thiazolo[5′,4′:4,5]pyrrolo[2,3-d]pyridazin-5(6H)-one, or simply as Compound 2.

In one aspect, as part of an eleventh embodiment, the compound administered to the subject (e.g., as in any one of the first to eighth embodiments), is of the structural formula:

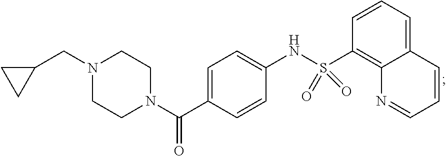 [SMILES: O=C(c1ccc(NS(=O)(=O)c2cccc3cccnc23)cc1)N1CCN(CC2CC2)CC1]

or a pharmaceutically acceptable salt thereof. This compound is also known as “mitapivat”, “AG-348”, or by its chemical name “N-(4-(4-(cyclopropylmethyl)piperazine-1-carbonyl)phenyl)quinoline-8-sulfonamide.” Alternatively, as part of an eleventh embodiment, mitapivat or a pharmaceutically acceptable salt thereof, is administered to the subject (e.g., as in any one of the first to eighth embodiments) in a crystalline form. In another alternative, as part of an eleventh embodiment, mitapivat, or a pharmaceutically acceptable salt thereof, is administered to the subject (e.g., as in any one of the first to eighth embodiments) as an amorphous form. In a further alternative embodiment, as part of an eleventh embodiment, mitapivat, or a pharmaceutically acceptable salt thereof, is administered to the subject (e.g., as in any one of the first to eighth embodiments) as a mixture of solid state forms (e.g., a mixture of one or more crystalline forms or a mixture of one or more crystalline forms and an amorphous form).

In one aspect, as part of a twelfth embodiment, the compound administered to the subject (e.g., as in any one of the first to eighth embodiments), is a pharmaceutically acceptable salt of mitapivat. Alternatively, as part of a twelfth embodiment, the compound administered to the subject (e.g., as in any one of the first to eighth embodiments), is a sulfate salt of mitapivat. In another alternative, as part of a twelfth embodiment, the compound administered to the subject (e.g., as in any one of the first to eighth embodiments), is a hemisulfate salt of mitapivat. In another alternative, as part of a twelfth embodiment, the compound administered to the subject (e.g., as in any one of the first to eighth embodiments), is a hydrated hemisulfate salt of mitapivat. In another alternative, as part of a twelfth embodiment, the compound administered to the subject (e.g., as in any one of the first to eighth embodiments), is a hemisulfate sesquihydrate salt of mitapivat, also known as mitapivat sulfate or 1-(cyclopropylmethyl)-4-(4-(quinoline-8-sulfonamido)benzoyl)piperazin-1-ium hemisulfate sesquihydrate having Formula A:

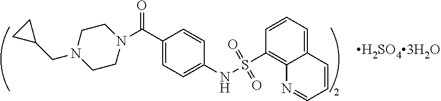

Formula A. In another alternative, as part of a twelfth embodiment, the compound administered to the subject (e.g., as in any one of the first to eighth embodiments), is a sulfate trihydrate salt, also referred to as (and is equivalent to) mitapivat trihydrate or 1-(cyclopropylmethyl)-4-(4-(quinoline-8-sulfonamido)benzoyl)piperazin-1-ium sulfate trihydrate having Formula B:

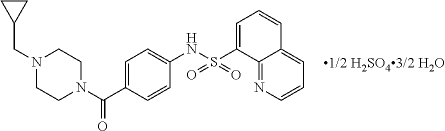 [SMILES: O=C(c1ccc(NS(=O)(=O)c2cccc3cccnc23)cc1)N1CCN(CC2CC2)CC1]

Formula B. The hemisulfate sesquihydrate salt of mitapivat (i.e., mitapivat sulfate) can be crystalline, for example, Form A as disclosed in U.S. Publication No. 20200277279. Form A is characterized by one or more of the following x-ray powder diffraction patterns at 2Θ angles (±0.2°) using Cu Kα radiation: 9.9°, 15.8°, and 22.6°; 15.0°, 17.1°, 21.3°, and 21.9°; 9.9°, 15.0°, 15.8°, 17.1°, 21.3°, 21.9°, and 22.6°; 9.9°, 11.4°, 15.0°, 15.3°, 15.8°, 17.1°, 17.7°, 21.3°, 21.9°, 22.6°, and 23.5°; or 4.9°, 9.9°, 11.0°, 11.4°, 11.7°, 12.3°, 12.8°, 13.6°, 13.9°, 14.2°, 15.0°, 15.3°, 15.8°, 17.1°, 17.4°, 17.7°, 18.8°, 19.1°, 19.8°, 21.3°, 21.9°, 22.6°, 23.0°, 23.2°, 23.50, 23.8°, 24.1°, 24.5°, 25.3°, 25.6°, 26.1°, 27.1°, 28.10, and 29.8°. In some embodiments, Form A is characterized by x-ray powder diffraction peaks at 2Θ angles (±0.2°) 9.9°, 15.8°, and 22.6°. In certain embodiments, Form A is characterized by x-ray powder diffraction peaks at 20 angles (±0.2°) 9.9°, 15.8°, and 22.6° and at least one additional x-ray powder diffraction peak at 2Θ angles (±0.2°) selected from 15.0°, 17.1°, 21.3°, and 21.9°. In certain embodiments, Form A is characterized by x-ray powder diffraction peaks at 2Θ angles (±0.2°) 9.9°, 15.8°, and 22.6°; and at least two additional x-ray powder diffraction peaks at 20 angles (±0.2°) selected from 15.0°, 17.1°, 21.3°, and 21.9°. In yet another alternative, Form A is characterized by x-ray powder diffraction peaks at 2Θ angles (±0.2°) 9.9°, 15.8°, and 22.6°; and at least three additional x-ray powder diffraction peaks at 2Θ angles (±0.2°) selected from 15.0°, 17.1°, 21.3°, and 21.9°. In certain embodiments, Form A is characterized by x-ray powder diffraction peaks at 2Θ angles (±0.2°) 9.9°, 15.0°, 15.8°, 17.1°, 21.3°, 21.9°, and 22.6°. In certain embodiments, Form A is characterized by x-ray powder diffraction peaks at 2Θ angles (±0.2°) 9.9°, 11.4°, 15.0°, 15.3°, 15.8°, 17.1°, 17.7°, 21.3°, 21.9°, 22.6°, and 23.5°. In certain embodiments, Form A is characterized by x-ray powder diffraction peaks at 2Θ angles (±0.2°) 4.9°, 9.9°, 11.0°, 11.4°, 11.7°, 12.3°, 12.8°, 13.6°, 13.9°, 14.2°, 15.0°, 15.3°, 15.8°, 17.1°, 17.4°, 17.7°, 18.8°, 19.1°, 19.8°, 21.3°, 21.9°, 22.6°, 23.0°, 23.2°, 23.5°, 23.8°, 24.1°, 24.5°, 25.3°, 25.6°, 26.1°, 27.1°, 28.1°, and 29.8°. In yet another alternative, Form A is characterized by a differential scanning calorimetry (DSC) thermograph comprising endotherm peaks at about 159° C.±5° C. and 199° C.±5° C. In yet another alternative, crystalline Form A is characterized by a thermogravimetric analysis (TGA) thermogram comprising a weight loss of about 4.5±0.5% up to 180° C.±2° C. In some embodiments, the hemisulfate sesquihydrate salt of mitapivat is 1-(cyclopropylmethyl)-4-(4-(quinoline-8-sulfonamido)benzoyl)piperazin-1-ium hemisulfate sesquihydrate Form A.

The term “amorphous” means a solid that is present in a non-crystalline state or form. Amorphous solids are disordered arrangements of molecules and therefore possess no distinguishable crystal lattice or unit cell and consequently have no definable long-range ordering. Solid state ordering of solids may be determined by standard techniques known in the art, e.g., by x-ray powder diffraction (XRPD) or differential scanning calorimetry (DSC). Amorphous solids can also be differentiated from crystalline solids, e.g., by birefringence using polarized light microscopy.

The 2-theta values of the x-ray powder diffraction patterns for the crystalline forms described herein may vary slightly from one instrument to another and also depending on variations in sample preparation and batch-to-batch variation due to factors such as temperature variation, sample displacement, and the presence or absence of an internal standard. Therefore, unless otherwise defined, the XRPD patterns/assignments recited herein are not to be construed as absolute and can vary±0.2 degrees. It is well known in the art that this variability will account for the above factors without hindering the unequivocal identification of a crystal form. Unless otherwise specified, the 2-theta values provided herein were obtained using Cu Kα1 radiation.

The compounds or pharmaceutically acceptable salts described herein, e.g., as in any one of the first to twelfth embodiments, may be formulated and administered as a pharmaceutical composition. Pharmaceutical compositions can be prepared by methods known in the art of pharmacology. In one aspect, the pharmaceutical compositions are orally administered in an orally acceptable dosage form including, but not limited to, granules or minitablets, capsules, tablets, emulsions and aqueous suspensions, dispersions and solutions.

In one aspect, as part of a thirteenth embodiment, a subject being treated by a disclosed compound, pharmaceutically acceptable salt, or composition (including those subjects and compounds described in any one of the first, second, and fourth through twelfth embodiments) experiences a hemoglobin response.

As used herein, “hemoglobin response” refers to an increase from a baseline Hb level (i.e., Hb concentration) of the subject, where the subject's hemoglobin response is measured over a period of time during treatment with a compound disclosed herein or a pharmaceutically acceptable salt or composition thereof or following administration of a compound disclosed herein or a pharmaceutically acceptable salt or composition thereof. Unless stated to the contrary, the terms “during treatment” or “following administration” when used in connection with a disclosed compound, pharmaceutically acceptable salt, or composition refers to ongoing treatment or administration (i.e., the subject will continue to be treated with or administered the disclosed compound, pharmaceutically acceptable salt, or composition thereof). The terms hemoglobin (Hb) level and hemoglobin concentration are used synonymously herein. As used herein, the term “baseline” refers to a level or concentration that is measured or established prior to treatment or during treatment with a compound disclosed herein or pharmaceutically acceptable salt or composition thereof. For example, as used herein the term “baseline hemoglobin level” refers to a subject's hemoglobin (Hb) level that is measured or established prior to treatment or during treatment with a compound disclosed herein or pharmaceutically acceptable salt or composition thereof. In one aspect, “hemoglobin response” refers to an increase from a baseline Hb level (i.e., Hb concentration) of the subject, where the subject's hemoglobin response is measured over a period of time during treatment. In another aspect, “hemoglobin response” refers to an increase from a baseline Hb level (i.e., Hb concentration) of the subject, where the subject's hemoglobin response is measured over a period of time following administration, e.g., for 1 week of treatment, 2 weeks of treatment, 3 weeks of treatment, 4 weeks of treatment, 3 months of treatment, 6 months of treatment or 1 year of treatment or longer.

In one aspect, as part of a fourteenth embodiment, the hemoglobin level of the subject being treated (including any one of the first through thirteenth embodiments) increases from baseline over a period of at least 1 week, at least 2 weeks, at least 3 weeks, at least 4 weeks, at least 6 weeks, at least 8 weeks, at least 10 weeks, at least 12 weeks, at least 14 weeks, at least 16 weeks, at least 18 weeks, at least 20 weeks, at least 30 weeks, at least 40 weeks, or at least 50 weeks during treatment with a disclosed compound, or pharmaceutically acceptable salt or composition thereof. In one aspect, as part of a fourteenth embodiment, the hemoglobin level of the subject being treated (including any one of the first through thirteenth embodiments) increases from baseline over a period of at least 1 week, at least 2 weeks, at least 3 weeks, at least 4 weeks, at least 6 weeks, at least 8 weeks, at least 10 weeks, at least 12 weeks, at least 14 weeks, at least 16 weeks, at least 18 weeks, at least 20 weeks, at least 30 weeks, at least 40 weeks, or at least 50 weeks during treatment with a disclosed compound, or pharmaceutically acceptable salt or composition thereof. Alternatively, as part of a fourteenth embodiment, the hemoglobin level of the subject being treated (including any one of the first through thirteenth embodiments) increases from baseline over a period of at least 4 weeks, at least 6 weeks, at least 8 weeks, at least 10 weeks, at least 12 weeks, at least 14 weeks, at least 16 weeks, at least 18 weeks, or at least 20 weeks during treatment with a disclosed compound, or pharmaceutically acceptable salt or composition thereof. In another alternative, as part of a fourteenth embodiment, the hemoglobin level of the subject being treated (including any one of the first through thirteenth embodiments) increases from baseline from week 1 through week 20, from week 1 through week 18, from week 1 through week 16, from week 4 through week 20, from week 4 through week 18, from week 4 through week 16, from week 6 through week 20, from week 6 through week 18, from week 6 through week 16, from week 8 through week 20, from week 8 through week 18, from week 8 through week 16, from week 10 through week 20, from week 10 through week 18, or from week 10 through week 16, during treatment with a disclosed compound, or pharmaceutically acceptable salt or composition thereof. In another alternative, as part of a fourteenth embodiment, the hemoglobin level of the subject being treated (including any one of the first through thirteenth embodiments) increases from baseline from week 8 through week 16 during treatment with a disclosed compound, or pharmaceutically acceptable salt or composition thereof. In another alternative, as part of a fourteenth embodiment, the hemoglobin level of the subject being treated (including any one of the first through thirteenth embodiments) increases from baseline at ≥2, ≥3, ≥4, ≥5, or ≥6 consecutive time points from week 1 through week 20, from week 1 through week 18, from week 1 through week 16, from week 4 through week 20, from week 4 through week 18, from week 4 through week 16, from week 6 through week 20, from week 6 through week 18, from week 6 through week 16, from week 8 through week 20, from week 8 through week 18, from week 8 through week 16, from week 10 through week 20, from week 10 through week 18, or from week 10 through week 16, during treatment with a disclosed compound, or pharmaceutically acceptable salt or composition thereof. In another alternative, as part of a fourteenth embodiment, the hemoglobin level of the subject being treated (including any one of the first through thirteenth embodiments) increases from baseline at ≥2 consecutive time points from week 8 through week 16 during treatment with a disclosed compound, or pharmaceutically acceptable salt or composition thereof. In another alternative, as part of a fourteenth embodiment, the hemoglobin level of the subject being treated (including any one of the first through thirteenth embodiments) increases from baseline for ≥1 consecutive week, for ≥2 consecutive weeks, for ≥3 consecutive weeks, for ≥4 consecutive weeks, for ≥5 consecutive weeks, for ≥6 consecutive weeks, for ≥7 consecutive weeks, for ≥8 consecutive weeks, for ≥9 consecutive weeks, or for ≥10 consecutive weeks during treatment with a disclosed compound, or pharmaceutically acceptable salt or composition thereof. In another alternative, as part of a fourteenth embodiment, the hemoglobin level of the subject being treated (including any one of the first through thirteenth embodiments) increases from baseline for ≥8 consecutive weeks during treatment with a disclosed compound or pharmaceutically acceptable salt or composition thereof.

In one aspect, as part of a fifteenth embodiment, the hemoglobin level of the subject being treated (including any one of the first through fourteenth embodiments) improves by at least 1.0 g/dL (e.g., ≥1.0 g/dL increase from baseline) during treatment with a disclosed compound, or pharmaceutically acceptable salt or composition thereof. Alternatively, as part of a fifteenth embodiment, the hemoglobin level of the subject being treated (including any one of the first through fourteenth embodiments) improves by at least 1.5 g/dL (e.g., ≥1.5 g/dL increase from baseline) during treatment with a disclosed compound, or pharmaceutically acceptable salt or composition thereof. In another alternative, as part of a fifteenth embodiment, the hemoglobin level of the subject being treated (including any one of the first through fourteenth embodiments) improves by at least 2.0 g/dL (e.g., ≥2.0 g/dL increase from baseline) during treatment with a disclosed compound, or pharmaceutically acceptable salt or composition thereof. In another alternative, as part of a fifteenth embodiment, the hemoglobin level of the subject being treated (including any one of the first through fourteenth embodiments) improves by more than 2.0 g/dL (e.g., ≥2.0 g/dL increase from baseline) during treatment with a disclosed compound, or pharmaceutically acceptable salt or composition thereof.

In one aspect, as part of a sixteenth embodiment, a subject being treated by a disclosed compound, or pharmaceutically acceptable salt or composition thereof (including any one of the first through fifteenth embodiments) is classified as a subject who is non-transfusion dependent (NTD) prior to treatment.

As used herein, the terms non-transfusion dependent (NTD) and “nontransfused” refers to those subjects who do not require regular blood transfusions prior to treatment with a disclosed compound or pharmaceutically acceptable salt or composition thereof. In one aspect, NTD refers to those subjects who have<3 red blood cell (RBC) units in the 16-week period before administration of the first dose of a disclosed compound, or pharmaceutically acceptable salt or composition thereof and no transfusions in the 8-week period before administration of the first dose of a disclosed compound, or pharmaceutically acceptable salt or composition thereof.

As used herein, the term transfusion dependent (TD) refers to those subjects who require regular blood transfusions.

In one aspect, as part of a seventeenth embodiment, a subject being treated by a disclosed compound or pharmaceutically acceptable salt or composition thereof (including any one of the first through fifteenth embodiments) becomes transfusion independent during treatment. In some aspects, as part of an seventeenth embodiment, a subject being treated by a disclosed compound or pharmaceutically acceptable salt or composition thereof (including any one of the first through fifteenth embodiments) becomes transfusion independent for a period of time during treatment.

As used herein, the terms “transfusion independent” or “transfusion independence” are used interchangeably herein and refer to those subjects that are free of (i.e., have not had) red blood cell transfusions for a period of time (also referred to herein as being “transfusion-free”). In some aspects, the terms “transfusion independent” or “transfusion independence” refer to subjects that have not had a red blood cell transfusion over a period of 16 consecutive weeks. In some aspects, subjects that have not had a red blood cell transfusion over a period of 16 consecutive weeks are referred to as being transfusion free. In other aspects, the terms “transfusion independent” or “transfusion independence” refer to subjects that have not had a red blood cell transfusion over a period of ≥8 consecutive weeks. In some aspects, subjects that have not had a red blood cell transfusion over a period of ≥8 consecutive weeks are referred to as being transfusion free.

In one aspect, as part of an eighteenth embodiment, a subject being treated by a disclosed compound or pharmaceutically acceptable salt or composition thereof (including any one of the first through fifteenth or seventeenth embodiments) is classified as having a high transfusion burden (HTB). Alternatively, as part of an eighteenth embodiment, a subject being treated by a disclosed compound or pharmaceutically acceptable salt or composition thereof (including any one of the first through fifteenth or seventeenth embodiments) is classified as having a high transfusion burden (HTB) prior to treatment. In another alternative, as part of an eighteenth embodiment, a subject being treated by a disclosed compound or pharmaceutically acceptable salt or composition thereof (including any one of the first through fifteenth or seventeenth embodiments) is classified as having a high transfusion burden (HTB) which is characterized by the subject receiving at least 8 red blood cell (RBC) units over the course of 16 weeks and having greater than or equal to 4 transfusion episodes over the course of 8 weeks. In another alternative, as part of an eighteenth embodiment, a subject being treated by a disclosed compound or pharmaceutically acceptable salt or composition thereof (including any one of the first through fifteenth or seventeenth embodiments) is classified as having a high transfusion burden (HTB) which is characterized by the subject receiving at least 8 RBC units over the course of 16 weeks and having greater than or equal to 4 transfusion episodes over the course of 8 weeks prior to treatment. In another alternative, as part of an eighteenth embodiment, a subject being treated by a disclosed compound or pharmaceutically acceptable salt or composition thereof (including any one of the first through fifteenth or seventeenth embodiments) is classified as having a high transfusion burden (HTB) which is characterized by the subject receiving at least 8 RBC units over the course of 16 weeks and receiving at least 4 RBC units over a period of 8 weeks during the course of the 16 weeks prior to treatment.

In one aspect, as part of a nineteenth embodiment, a subject being treated by a disclosed compound or pharmaceutically acceptable salt or composition thereof (including any one of the first through fifteenth or seventeenth embodiments) is classified as having a low transfusion burden (LTB). Alternatively, as part of a nineteenth embodiment, a subject being treated by a disclosed compound or pharmaceutically acceptable salt or composition thereof (including any one of the first through fifteenth or seventeenth embodiments) is classified as having a low transfusion burden (LTB) prior to treatment. In another alternative, as part of a nineteenth embodiment, a subject being treated by a disclosed compound or pharmaceutically acceptable salt or composition thereof (including any one of the first through fifteenth or seventeenth embodiments) is classified as having a low transfusion burden (LTB) which is characterized by the subject receiving between 3-7 RBC units over the course of 16 weeks in at least 2 transfusion episodes with a maximum of 3 transfusion episodes over the course of 8 weeks prior to treatment. In another alternative, as part of a nineteenth embodiment, a subject being treated by a disclosed compound or pharmaceutically acceptable salt or composition thereof (including any one of the first through fifteenth or seventeenth embodiments) is classified as having a low transfusion burden (LTB) which is characterized by the subject receiving between 3-7 RBC units over the course of 16 weeks in at least 2 transfusion episodes with a maximum of 3 transfusion episodes over the course of 8 weeks at some point during treatment.

In one aspect, as part of a twentieth embodiment, a subject being treated by a disclosed compound or pharmaceutically acceptable salt or composition thereof (including any one of the first through twenty-first embodiments) becomes transfusion independent for ≥1 consecutive week, for ≥2 consecutive weeks, for ≥3 consecutive weeks, for ≥4 consecutive weeks, for ≥5 consecutive weeks, for ≥6 consecutive weeks, for ≥7 consecutive weeks, for ≥8 consecutive weeks, for ≥9 consecutive weeks, or for ≥10 consecutive weeks during treatment. Alternatively, as part of a twentieth embodiment, a subject being treated by a disclosed compound or pharmaceutically acceptable salt or composition thereof (including any one of the first through twenty-first embodiments) becomes transfusion independent for ≥8 consecutive weeks during treatment. In another alternative, as part of a twentieth embodiment, a subject being treated by a disclosed compound or pharmaceutically acceptable salt or composition thereof (including any one of the first through twenty-first embodiments) becomes transfusion independent for ≥8 consecutive weeks during treatment, wherein the subject is classified as having a low transfusion burden (LTB) prior to treatment.

In one aspect, as part of a twenty-first embodiment, a subject being treated by a disclosed compound or pharmaceutically acceptable salt or composition thereof (including any one of the first through twentieth embodiments) experiences a change from baseline in total transfused red blood cell (RBC) units during treatment. In one aspect, as part of a twenty-first embodiment, a subject being treated by a disclosed compound or pharmaceutically acceptable salt or composition thereof (including any one of the first through twentieth embodiments) experiences a reduction in total transfused red blood cell (RBC) units during treatment in comparison to the subject's baseline total transfused red blood cell (RBC) units.

In one aspect, as part of a twenty-second embodiment, a subject being treated by a disclosed compound or pharmaceutically acceptable salt or composition thereof (including any one of the first through twenty-first embodiments) experiences a change from baseline in total transfused red blood cell (RBC) units which is characterized by a ≥10%, ≥20%, ≥30%, ≥40%, ≥50%, ≥60%, ≥70%, or ≥80% reduction in total transfused RBC units during treatment. Alternatively, as part of a twenty-second embodiment, a subject being treated by a disclosed compound or pharmaceutically acceptable salt or composition thereof (including any one of the first through twenty-first embodiments) experiences a change from baseline in total transfused red blood cell (RBC) units characterized by a ≥10%, ≥20%, ≥30%, ≥40%, ≥50%, ≥60%, ≥70%, or ≥80% reduction in total transfused RBC units over a period of ≥1 consecutive week, ≥2 consecutive weeks, ≥3 consecutive weeks, ≥4 consecutive weeks, ≥5 consecutive weeks, ≥6 consecutive weeks, ≥7 consecutive weeks, ≥8 consecutive weeks, ≥9 consecutive weeks, or ≥10 consecutive weeks during treatment. In another alternative, as part of a twenty-second embodiment, a subject being treated by a disclosed compound or pharmaceutically acceptable salt or composition thereof (including any one of the first through twenty-first embodiments) experiences a change from baseline in total transfused red blood cell (RBC) units characterized by a ≥50% reduction in total transfused RBC units during treatment. In another alternative, as part of a twenty-second embodiment, a subject being treated by a disclosed compound or pharmaceutically acceptable salt or composition thereof (including any one of the first through twenty-first embodiments) experiences a change from baseline in total transfused red blood cell (RBC) units characterized by a ≥50% reduction in total transfused RBC units over a period of ≥1 consecutive week, ≥2 consecutive weeks, ≥3 consecutive weeks, ≥4 consecutive weeks, ≥5 consecutive weeks, ≥6 consecutive weeks, ≥7 consecutive weeks, ≥8 consecutive weeks, ≥9 consecutive weeks, or ≥10 consecutive weeks during treatment. In another alternative, as part of a twenty-second embodiment, a subject being treated by a disclosed compound or pharmaceutically acceptable salt or composition thereof (including any one of the first through twenty-first embodiments) experiences a change from baseline in total transfused red blood cell (RBC) units characterized by a ≥50% reduction in total transfused RBC units over a period of ≥8 consecutive weeks during treatment. In another alternative, as part of a twenty-second embodiment, a subject being treated by a disclosed compound or pharmaceutically acceptable salt or composition thereof (including any one of the first through twenty-first embodiments) experiences a change from baseline in total transfused red blood cell (RBC) units characterized by a ≥50% reduction in total transfused RBC units over a period of ≥8 consecutive weeks during treatment, wherein the subject is classified as having a high transfusion burden (HTB) prior to treatment.

In one aspect, as part of a twenty-third embodiment, a subject being treated by a disclosed compound or pharmaceutically acceptable salt or composition thereof (including any one of the first through twenty-second embodiments) experiences a reduction in 2,3-diphosphoglycerate (2,3-DPG) concentration during treatment as compared to the subject's baseline 2,3-DPG concentration.

In one aspect, as part of a twenty-fourth embodiment, a subject being treated by a disclosed compound or pharmaceutically acceptable salt or composition thereof (including any one of the first through twenty-third embodiments) experiences an increase in adenosine triphosphate (ATP) concentration during treatment (as compared to the subject's baseline ATP concentration). Alternatively, as part of a twenty-fourth embodiment, a subject being treated by a disclosed compound or pharmaceutically acceptable salt or composition thereof (including any one of the first through twenty-third embodiments) experiences an increase in adenosine triphosphate (ATP) concentration of more than 50%, more than 55%, more than 60%, more than 65%, more than 70%, and more than 75% during treatment (as compared to the subject's baseline ATP concentration). In another alternative, as part of a twenty-fourth embodiment, a subject being treated by a disclosed compound or pharmaceutically acceptable salt or composition thereof (including any one of the first through twenty-third embodiments) experiences an increase in adenosine triphosphate (ATP) concentration of 68%, 71%, or 74% during treatment (as compared to the subject's baseline ATP concentration). In another alternative, as part of a twenty-fourth embodiment, a subject being treated by a disclosed compound or pharmaceutically acceptable salt or composition thereof (including any one of the first through twenty-third embodiments) experiences an increase in adenosine triphosphate (ATP) concentration of more than 50%, more than 55%, more than 60%, more than 65%, more than 70%, and more than 75% after 10, after 20, after 30, after 40, after 50, after 60, or after 65 days of daily dosing (as compared to the subject's baseline ATP concentration). In another alternative, as part of a twenty-fourth embodiment, a subject being treated by a disclosed compound or pharmaceutically acceptable salt or composition thereof (including any one of the first through twenty-third embodiments) experiences an increase in adenosine triphosphate (ATP) concentration of 68% after 56 days of daily dosing, 71% after 56 days of daily dosing, or 74% after 56 days of daily dosing (as compared to the subject's baseline ATP concentration).

In one aspect, as part of a twenty-fifth embodiment, the MDS described herein (including any one of the first to sixth and tenth to twenty-fourth embodiments) is low risk MDS as characterized by the Revised International Prognostic Scoring System (IPSS-R) for MDS. See, for example, Greenberg P L, Tuechler H, Schanz J, et al. Revised International Prognostic Scoring System for myelodysplastic syndromes. Blood. 2012; 120:2454-2465. Low risk MDS includes, for example, an IPSS-R score of greater than 1.5 to 3. Alternatively, as part of a twenty-fifth embodiment, the MDS described herein (including any one of the first to sixth and tenth to twenty-fourth embodiments) is very low risk MDS as characterized by the Revised International Prognostic Scoring System (IPSS-R) for MDS. Very low risk MDS includes, for example, an IPSS-R score of less than or equal to 1.5. In another alternative, as part of a twenty-fifth embodiment, the MDS described herein (including any one of the first to sixth and tenth to twenty-fourth embodiments) is intermediate risk MDS as characterized by the Revised International Prognostic Scoring System (IPSS-R) for MDS. Intermediate risk MDS includes, for example, an IPSS-R score of greater than 3 to 4.5. In still another alternative, as part of a twenty-fifth embodiment, the MDS described herein (including any one of the first to sixth and tenth to twenty-fourth embodiments) the term “lower risk MDS” encompasses very low risk MDS and low risk MDS as described above.

In one aspect, as part of a twenty-sixth embodiment, the subject described herein (including any one of the first to twenty-fifth embodiments) is male. Alternatively, as part of a twenty-sixth embodiment, the subject described herein (including any one of the first to twenty-fifth embodiments) is female. Alternatively, as part of a twenty-sixth embodiment, the subject described herein (including any one of the first to twenty-fifth embodiments) is an adult male. Alternatively, as part of a twenty-sixth embodiment, the subject described herein (including any one of the first to twenty-fifth embodiments) is an adult female. In another alternative, as part of a twenty-sixth embodiment, the subject described herein (including any one of the first to twenty-fifth embodiments) is a male child or a female child. Alternatively, as part of a twenty-sixth embodiment, the subject described herein (including any one of the first to twenty-fifth embodiments) is an adult (i.e., ≥18 years of age). In yet another alternative, as part of a twenty-sixth embodiment, the subject described herein (including any one of the first to twenty-fifth embodiments) is a child (i.e., <18 years of age).

In one aspect, as part of a twenty-seventh embodiment, a subject described herein (including any one of the first to twenty-sixth embodiments) is administered a therapeutically effective amount of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one, or a pharmaceutically acceptable salt thereof. Alternatively, as part of a twenty-seventh embodiment, a subject described herein (including any one of the first to twenty-sixth embodiments) is administered a composition comprising a therapeutically effective amount of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one, or a pharmaceutically acceptable salt thereof and a pharmaceutically acceptable carrier.

In one aspect, as part of a twenty-eighth embodiment, a therapeutically effective amount of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) is 2 mg daily, 3 mg daily, or 5 mg daily. Alternatively, as part of a twenty-eighth embodiment, a therapeutically effective amount of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) is 2 mg administered once daily (QD), 3 mg administered QD, or 5 mg administered QD. In another alternative, as part of a twenty-eighth embodiment, a therapeutically effective amount of a pharmaceutically acceptable salt of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one) disclosed herein (including any one of the first to twenty-seventh embodiments) is an amount that is equivalent to 2 mg daily, 3 mg daily, or 5 mg daily of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one as a free base. In another alternative, as part of a twenty-eighth embodiment, a therapeutically effective amount of a pharmaceutically acceptable salt of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) is an amount that is equivalent to 2 mg administered once daily (QD), 3 mg administered QD, or 5 mg administered QD of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one as a free base. In another alternative, as part of a twenty-eighth embodiment, a therapeutically effective amount of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) ranges from 0.5 mg to 10 mg QD. In another alternative, as part of a twenty-eighth embodiment, a therapeutically effective amount of a pharmaceutically acceptable salt of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) is an amount that is equivalent to a range of from 0.5 mg to 10 mg QD of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one as a free base. In another alternative, as part of a twenty-eighth embodiment, a therapeutically effective amount of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) ranges from 0.5 mg to 10 mg twice daily (BID). In another alternative, as part of a twenty-eighth embodiment, a therapeutically effective amount of a pharmaceutically acceptable salt of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one disclosed herein (including any one of the first to twenty-seventh embodiments) is an amount that is equivalent to a range of from 0.5 mg to 10 mg BID of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one as a free base. In another alternative, as part of a twenty-eighth embodiment, a therapeutically effective amount of Compound 1 disclosed herein (including any one of the first to twenty-seventh embodiments) ranges from 0.25 mg to 15 mg daily. In another alternative as part of a twenty-eighth embodiment, a therapeutically effective amount of a pharmaceutically acceptable salt of Compound 1 disclosed herein (including any one of the first to twenty-seventh embodiments) is an amount that is equivalent to a range of from 0.25 mg to 15 mg daily as a free base. In another alternative, as part of a twenty-eighth embodiment, a therapeutically effective amount of Compound 1 disclosed herein (including any one of the first to twenty-seventh embodiments) ranges from 0.25 mg to 2 mg QD or BID or from 1.5 mg to 5.5 mg QD or BID or from 4 mg to 6 mg QD or BID. In another alternative as part of a twenty-eighth embodiment, a therapeutically effective amount of a pharmaceutically acceptable salt of Compound 1 disclosed herein (including any one of the first to twenty-seventh embodiments) is an amount that is equivalent to a range of from 0.25 mg to 2 mg QD or BID or from 1.5 mg to 5.5 mg QD or BID or from 4 mg to 6 mg QD or BID as a free base. In another alternative, as part of a twenty-eighth embodiment, a therapeutically effective amount of Compound 1 disclosed herein (including any one of the first to twenty-seventh embodiments) is 1 mg or 5 mg QD or BID. In another alternative as part of a twenty-eighth embodiment, a therapeutically effective amount of a pharmaceutically acceptable salt of Compound 1 disclosed herein (including any one of the first to twenty-seventh embodiments) is an amount that is equivalent to 1 mg or 5 mg QD or BID as a free base.

In one aspect, as part of a twenty-ninth embodiment, a subject described herein, (including any one of the first to twenty-eighth embodiments) is treated (i.e., administered a compound, pharmaceutically acceptable salt, or composition described herein) for a period of at least 12 weeks, at least 14 weeks, at least 16 weeks, at least 24 weeks, at least 30 weeks, or at least 6 weeks. Alternatively, as part of a twenty-ninth embodiment, a subject described herein (including any one of the first to twenty-eighth embodiments) is treated (i.e., administered a compound, pharmaceutically acceptable salt, or composition described herein) for a period of up to 12 weeks, up to 14 weeks, up to 16 weeks, up to 24 weeks, up to 30 weeks, up to 36 weeks, up to 50 weeks, up to 100 weeks, or up to 160 weeks. In another alternative, as part of a twenty-ninth embodiment, subjects described herein (including any one of the first to twenty-eighth embodiments) are treated (i.e., administered a compound, pharmaceutically acceptable salt, or composition described herein) for a period of 16 weeks, of 24 weeks, or of 156 weeks. In another alternative, as part of a twenty-ninth embodiment, subjects described herein (including any one of the first to twenty-eighth embodiments) are treated (i.e., administered a compound, pharmaceutically acceptable salt, or composition described herein) indefinitely or for the remainder of the subject's life.

In one aspect, provided is the use of one or more of the disclosed pyruvate kinase activators or a pharmaceutically acceptable salt or composition thereof, for the manufacture of a medicament for treating a disclosed condition (e.g., as in any one of the embodiments disclosed above). In another aspect, also provided is the use of one or more of the disclosed pyruvate kinase activators or a pharmaceutically acceptable salt or composition thereof, for treating a disclosed condition (e.g., as in any one of the embodiments disclosed above).

Further details are described in the Exemplification section below and are included as part of the present invention.

### EXEMPLIFICATION

### Preparation of Compounds

### Example 1: Preparation of 2-((1H-pyrazol-3-yl)methyl)-6-((6-aminopyridin-2-yl)methyl)-4-methyl-4H-thiazolo[5′,4′:4,51 pyrrolo[2,3-d]pyridazin-5(6H)-one (Compound 1)

2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one can be prepared following the procedures outline for compound E8-4 of Example 8A in U.S. Pat. No. 11,040,036, the entire contents of which are incorporated herein by reference. Pharmaceutically acceptable salts of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one can also be prepared in view of the disclosure of U.S. Pat. No. 11,040,036.

2-((1H-pyrazol-3-yl)methyl)-4-methyl-6-((1-methyl-1H-pyrazol-3-yl)methyl)-4H-thiazolo[5′,4′:4,5]pyrrolo[2,3-d]pyridazin-5(6H)-one can be prepared following the procedures outline for compound E8-41 in U.S. Pat. No. 11,040,036, the entire contents of which are incorporated herein by reference. Pharmaceutically acceptable salts of 2-((1H-pyrazol-3-yl)methyl)-4-methyl-6-((1-methyl-1H-pyrazol-3-yl)methyl)-4H-thiazolo[5′,4′:4,5]pyrrolo[2,3-d]pyridazin-5(6H)-one can also be prepared in view of the disclosure of U.S. Pat. No. 11,040,036.

Mitapivat can be prepared according to the procedure outlined for compound VIII-8 in WO 2011/002817, the entire contents of which are incorporated herein by reference. Additional methods, salts, and crystalline forms can also be found in WO 2016/201227, and salts and crystalline forms can be found in WO 2019/104134 and WO 2020/237047, each of which are incorporated herein by reference in their entirety.

### MDS-Associated Anemia Mouse Models

In vivo mouse studies were conducted to determine the impacts of mitapivat and Compound 1 on the maturation of erythroblast populations under conditions of ineffective erythropoiesis comparable to that of MDS patients. To accomplish this, two established mouse models demonstrated to have ineffective erythropoiesis were used. To determine the impacts of mitapivat and Compound 1 under these conditions, bone marrow aspirates from diseased mice were collected and populations of erythroblasts were evaluated at various stages of maturation using flow cytometry. Hemoglobin (Hb), red blood cell (RBC) counts, and reticulocyte fractions were also monitored every 4 weeks in whole blood on a Sysmex XN-1000™ hematology analyzer to determine if disease burden and impact on erythroblast maturation could be detected in the periphery. Pharmacodynamics and pharmacokinetic analysis were also performed to confirm appropriate exposure and target engagement of the compounds (data not shown).

### Example 2: Study of mitapivat in Polg^{D257A} Mouse Model

Mitochondrial disruption has been linked to the development of anemia in MDS patients (see, e.g., Chen et al. Blood. 2009; 114(19):4045-53). Mitochondria play a key role in regulation of apoptosis, which is a mechanism highlighted in defunct hematopoiesis of MDS (Green et al., Science 1998; 281(5381):1309-1312, Kerbauy et al., Exp Hematol. 2007; 35(11): 1739-1746; Ahlqvist et al. Nature Communications. 2015; 6:6494). B6.12957(Cg)-Polg^{tm1prol}/J, or Polg^{D257A}, is a mouse model with a mutation in the N-terminus of the DNA polymerase γ gene that results in impaired proofreading activity in the mitochondria, leading to increased mtDNA mutagenesis (Jackson Laboratory #017341). See, Chen et al. Blood. 2009; 114(19):4045-53 and Ahlqvist et al. Nature Communications. 2015; 6:6494. Accumulation of these mutations leads to deregulated hematopoiesis and elevated apoptosis, comparable to mechanism of disease seen in MDS patients (Chen et al. Blood. 2009; 114(19):4045-53). Additionally, several lines of evidence demonstrate acquired pyruvate deficiency in MDS, implying that PKR may be a potential therapeutic target in this indication (Bovin P et al. British Journal of hematology. 1975; 18(3): 175-87; Valentine WN et al. Blood. 1973; 41(6):857-75; Arnold H et al. Clinica Chimica Acta. 1974; 57:187-9; Lin G et al. Chin J Hematol. 1997; 18(7):350-3). Here, we used the Polg^{D257A} mouse model to evaluate the therapeutic benefit of a PK activator, mitapivat, in this mechanism of anemia.

Male and female Polg^{D257A} mice were administered 200 mg/kg/day mitapivat in chow, ad libitum, for 18 weeks starting at 7.5 months of age, once anemic phenotype was established by hematology analysis. Whole blood was collected via tail vein into EDTA tubes and analyzed using a Sysmex XN-2000™ hematology system according to manufacturer protocol. Hemoglobin, red blood cells (RBCs), and reticulocytes were monitored every 2-4 weeks to track disease progression and severity. After 18 weeks of mitapivat treatment, RBC counts were elevated by 45% and hemoglobin levels were increased by 2.4 g/dL in mitapivat treated mice compared to untreated mutants. Complimentary to this, the frequency of reticulocytes was decreased by 30% in treated mice, suggesting improved erythropoiesis over untreated mice.

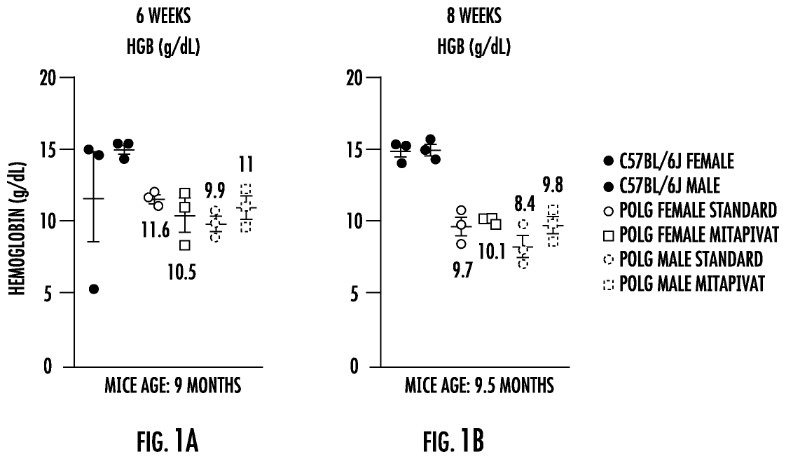

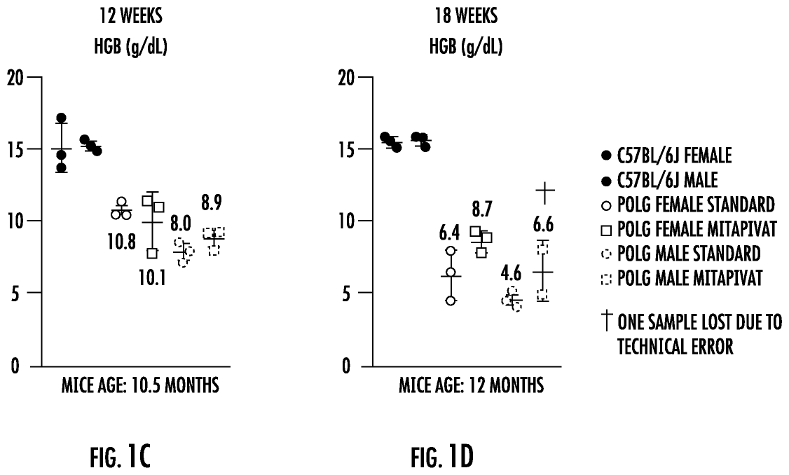

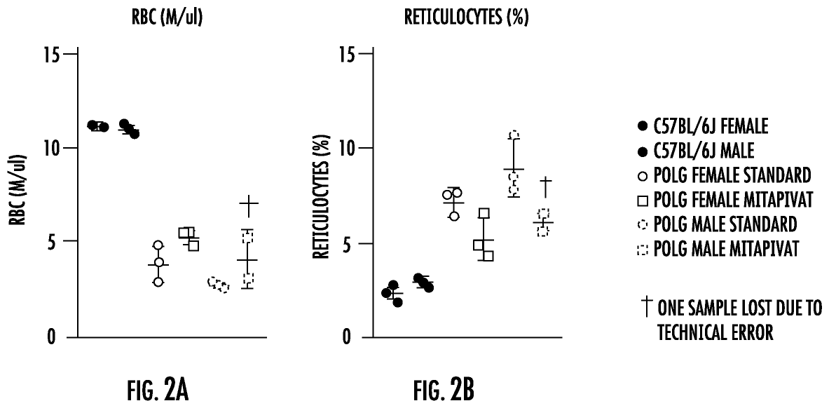

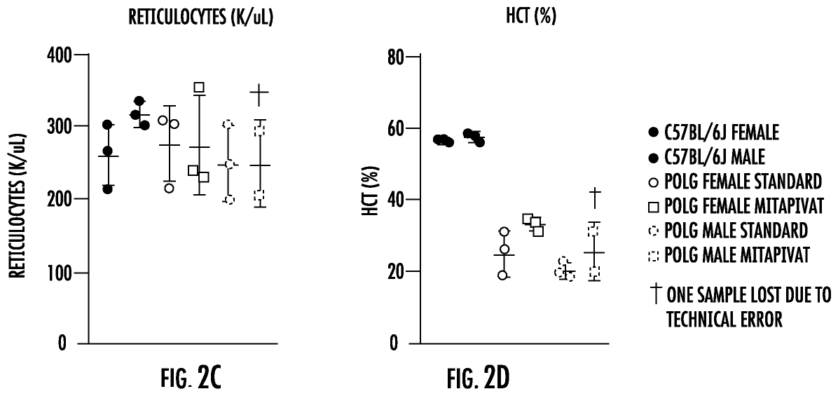

See FIGS. 1A-D and FIGS. 2A-D (C57BL/6J mice were used as controls). Interestingly, male mutators showed signs of therapeutic benefit starting at 6-weeks post-treatment with a 1-2 g/dL increase in hemoglobin, whereas female mutators responded after 18 weeks. Commensurate with increased hemoglobin was a marked decrease (about 30%) in reticulocyte count. See FIG. 2C.

### Example 3: Study of mitapivat and Compound 1 in Polg^{D257A} Mouse Model

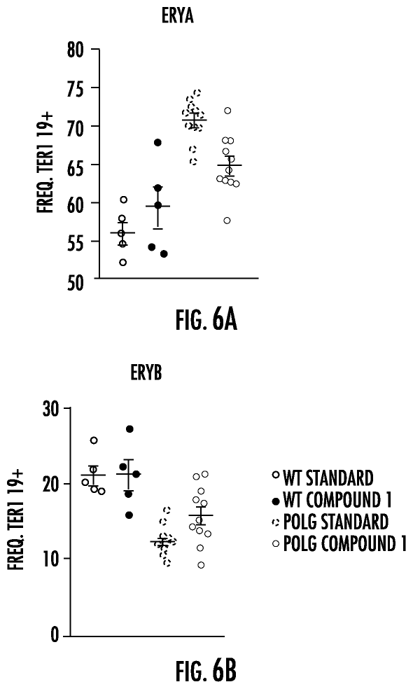

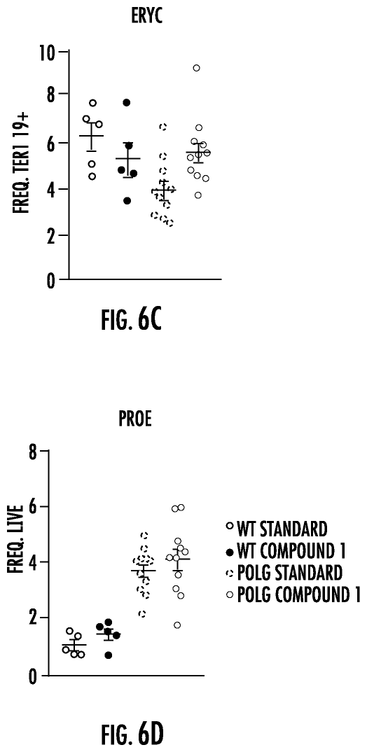

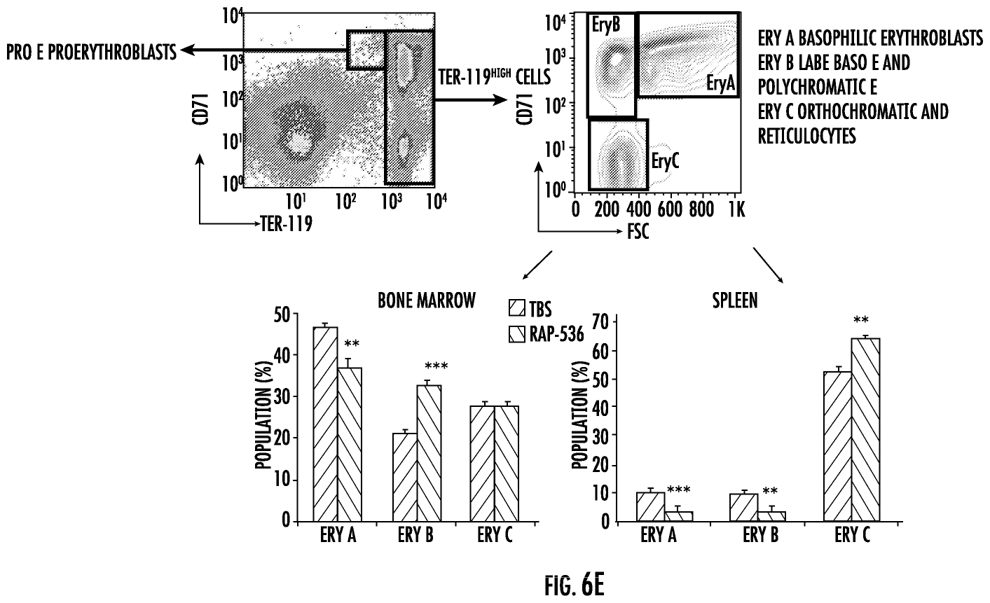

A second experiment was performed using the Polg^{D257A} model (RRID:IMSR_JAX:017341) from Example 2. In this experiment 20 mice (10 males and 10 females per group) were dosed with either mitapivat or Compound 1 in chow as described in Example 2. For the mitapivat group, the compound was dosed at 200 mg/kg/day and for the Compound 1 group, Compound 1 was dosed at 10 mg/kg/day. Treatment was started when the mice were 4 months of age and was continued for 8 months (rolling enrollment). Following 8 months of treatment (at 12 months of age), an evaluation of bone marrow aspirates was conducted by flow cytometry using well established markers of erythropoiesis following a modified procedure outlined in Suragani et al., Nat Med 2014 Apr; 20(4):408-14. Bone marrow aspirates were treated with ACK lysis buffer for 3 minutes to remove red blood cells from the sample. Remaining cells were washed and maintained in PBS+5% FBS throughout the remainder of the procedure. The flow cytometry panel included live/dead (Indol), B220 (BV421), CD5 (BV421), CD71 (AF700), and Terl19 (PE) antibodies. The gating strategy used for analysis was as follows: Live/B220-CD5-/Terl19+. Ter119+cells were then gated into populations using CD71 and FSC as outlined in the literature (Suragani et al., Nat Med 2014 Apr; 20(4):408-14; FIG. 6E). As seen in the bone marrow flow cytometry data shown in FIGS. 6A-D, treatment with Compound 1 resulted in a reduction in basophilic erythroblasts (EryA) coupled with an increase in late basophilic and polychromatic erythroblasts (EryB) as well as in orthochromatic and reticulocytes (EryC), suggesting treatment with Compound 1 improved erythroblast maturation in bone marrow of Polg^{D257A} mice. This evidence suggests an improvement of erythropoiesis with Compound 1 treatment in the Polg^{D257A} model. An increase in the ProE (proerythrocyte) population in Polg^{D257A} mice was also observed, however, there was no additional impact on the ProE population with Compound 1 treatment (as compared to vehicle control Polg^{D257A} mice). FIG. 6D. Flow cytometry data on the mitapivat treated Polg^{D257A} mice is not available as the mitapivat treatment arm is not yet completed.

Analysis of Hb, RBC, and reticulocytes in whole blood demonstrated that a significant disease burden was established in this model, with vehicle control Polg^{D257A} mice having an average Hb concentration of 7.9 g/dL compared to 13.9 g/dL in wild-type control mice. Data not shown. After 24 weeks (6 months thus far) of treatment, mitapivat increased Hb and RBC counts by 7% in Polg^{D257A} mice, with no change in reticulocytes. Data not shown. After 32 weeks of treatment with Compound 1 no change in Hb, RBC count, or reticulocyte fraction was observed. Data not shown.

The Polg^{D257A} model also supports the use of the disclosed PK activators in the treatment of diseases or disorders related to mitochondrial dysfunction. Although red blood cells are devoid of mitochondria, they are retained late in the process of hematopoiesis. Thus, the effects of PK activators on erythropoiesis and increase hemoglobin production are a result of PK activation in blood cell progenitor stem cells. The Polg^{D257A} model has a proofreading-defective mitochondrial DNA polymerase and has a dilatory effect on hematopoietic stem cell (HSC) differentiation (See Cell Stem Cell Volume 8, Issue 5, 6 May 2011, Pages 499-510). The consequence of rapidly accumulating mitochondrial DNA mutations causes progenitor stem cell differentiation and the loss of downstream progenitors. These deficits are likely attributable to reduced ATP generation from the dysfunctional mitochondria. Since HSCs express both PKR and PKM2 during differentiation, and since the disclosed PK activators enhance the function of both isozymes, the increase in ATP generation from the glycolytic pathway compensates for the loss of ATP generation in cells with mitochondrial dysfunction. The improvements in HSC multilineage stem cell differentiation demonstrated by PK activators point to the potential opportunity for PK activators to support somatic stem cell differentiation in patients where mitochondrial function may be compromised.

### Example 4: NHD13 Mouse Model

A NUP98-HOXD13 (NHD13) transgenic MDS associated mouse model will also be used to assess the therapeutic benefit of disclosed compounds. See e.g., Lin et al., Blood 2005 Jul 1;106(1):287-95 and Suragani et al., Nat Med 2014 Apr;20(4):408-14. Male and female NHD13 mice aged to 4 or 10 months will be administered a disclosed compound in chow, ad libitum (e.g., mitapivat or Compound 1) daily. A full blood panel and erythroid precursor analysis will be conducted after 2 months of treatment, and results will be analyzed.

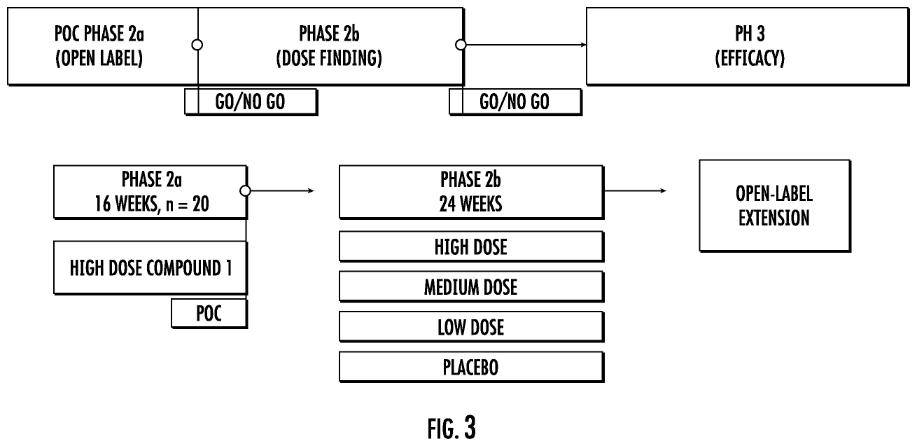

Evidence on the effects of the disclosed PKR activators on ineffective erythropoiesis that can be expected to translate to MDS (e.g., very low-risk, low-risk and intermediate risk MDS) includes: features of ineffective erythropoiesis are similar between thalassemia and MDS; PKR activators can improve survival and differentiation of erythroid cells in bone marrow; and PKR activators improves RBC functionality via increasing energy ATP, nucleotide biosynthesis, and antioxidative-stress responses via activating glycolysis. A proposed human clinical study is illustrated by FIG. 3.

### Example 5: Study of mitapivat or Compound 1 in NHD13 Mouse Model (age 4 months)

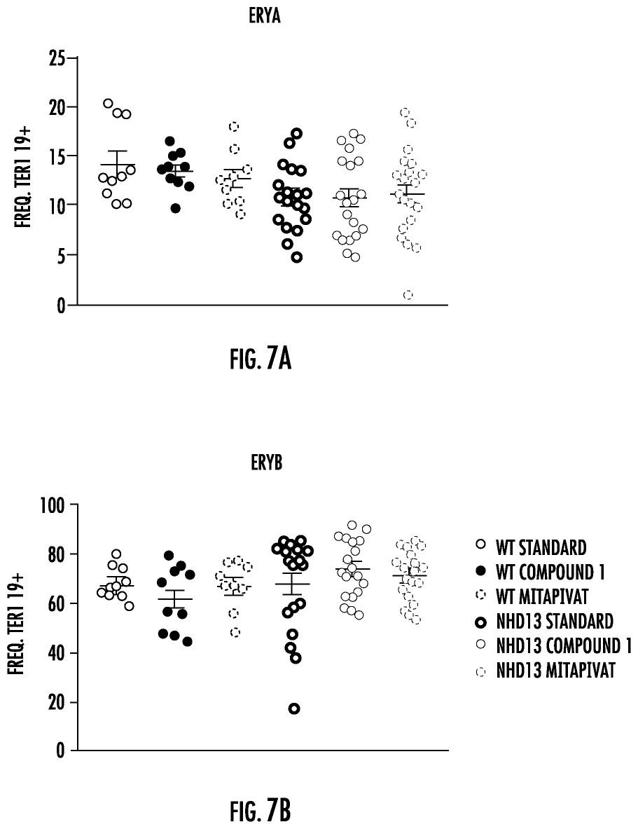

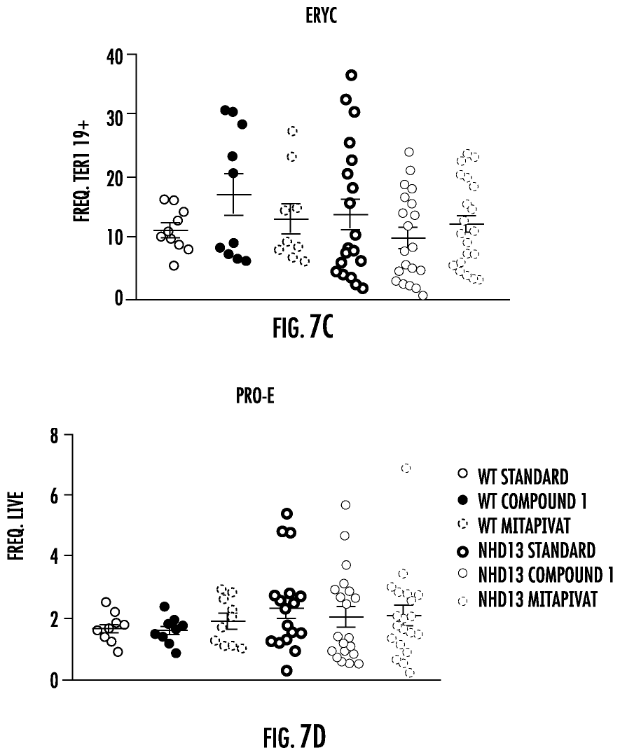

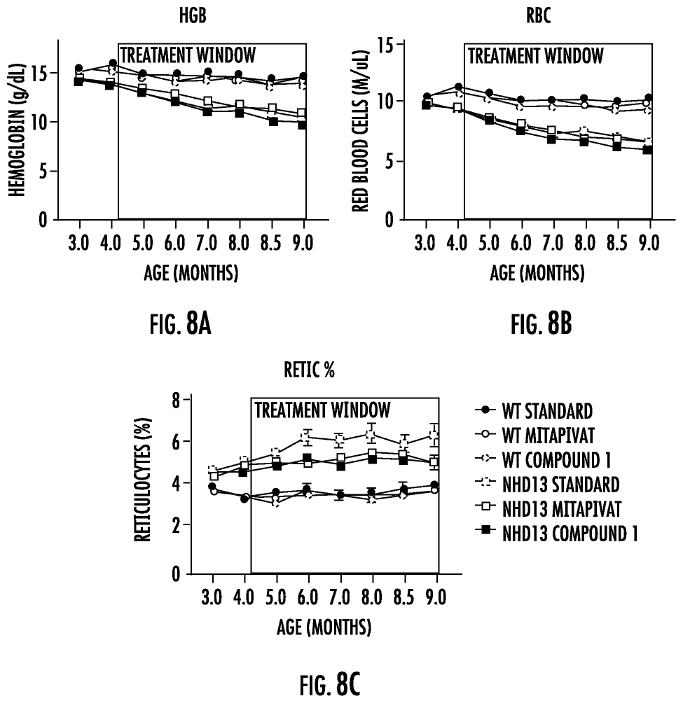

Using Example 4, 20 NHD13 mice (10 males and 10 females per group; RRID:IMSR_JAX:010505) aged 4 months were randomized into treatment groups using matched distribution of hemoglobin levels. Mice were dosed with either mitapivat or Compound 1 for 20 weeks in the same manner (in chow, ad libitum) as Example 3. After 20 weeks of treatment with either mitapivat or Compound 1, bone marrow aspirates of the NHD13 mice were collected as described above and analyzed by flow cytometry. Results from an evaluation of erythroblast populations in the bone marrow showed very little difference between erythroblast populations from wild type and NHD13 vehicle control mice. In addition, a significant decline in survival of vehicle control NHD13 mice as previously reported (Lin et al., Neoplasia 2005 July;106(1)) was not observed. Together, this data suggests the NHD13 model produced a milder form of disease than as described in the literature, complicating the assessment of treatment effects. As a result, it was difficult to detect any therapeutic benefit from the administration of either mitapivat or Compound 1 in the flow cytometry data (FIGS. 7A-D). This mild state of disease was also supported in whole blood analysis. At nine months of age, NHD13 mice on standard chow (vehicle control) had an average hemoglobin level of 10.4 g/dL, only 4 g/dL below wild-type controls. Evaluating the effect of treatments in NHD13 mice, mitapivat and Compound 1 both demonstrated a significant reduction in reticulocyte fraction (20%). (FIG. 8C) Treatment with mitapivat or Compound 1 also demonstrated a significant reduction in reticulocyte count (mitapivat by 15% and Compound 1 by 25%). Data not shown. These data demonstrate improvement in erythropoiesis of NHD13 mice with treatment, however minimal response in Hb or RBC count was observed compared to progressive anemia with vehicle controls (FIGS. 8A-B).

### Example 6: Study of mitapivat or Compound 1 in NHD13 Mouse Model (age 10 months)

A second experiment in NHD13 mice that are 10 months of age can also be conducted with the expectation that the disease burden will be more advanced and likely detectable in bone marrow by flow cytometry as described in Example 5. For this study, NHD13 mice can be randomized into treatment groups using matched distribution of hemoglobin levels at 10 months of age with 20 mice per treatment group (10 males and 10 females). Either mitapivat and/or Compound 1 can be dosed in chow given ad libitum, as described in Example 5, for 8 weeks. Hemoglobin, red blood cell counts, and reticulocyte fractions can be monitored every 4 weeks. The terminal collection, including bone marrow flow cytometry analysis, can occur at 12 months of age.

### Phase 2a/2b, Study of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one in Subject with Anemia Due to Lower-Risk Myelodysplastic Syndromes

A Phase 2a/2b multicenter study evaluating the efficacy and safety of 2-((1H-pyrazol-3-yl)methyl)-6-((6-aminopyridin-2-yl)methyl)-4-methyl-4H-thiazolo[5′,4′:4,5]pyrrolo[2,3-d]pyridazin-5(6H)-one, referred to herein as Compound 1 in subjects with anemia due to lower-risk myelodysplastic syndromes (LR-MDS) will be conducted.

### Study Design

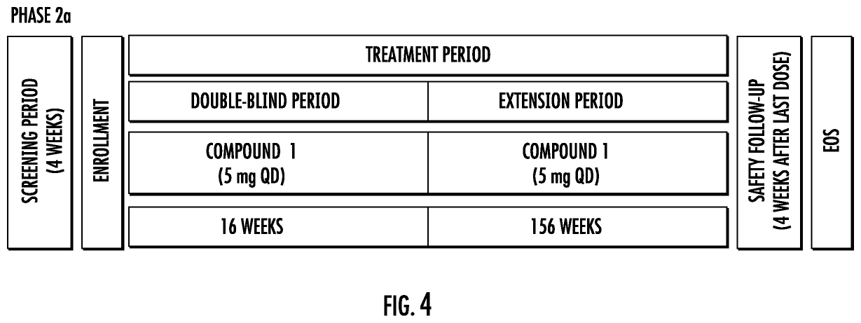

The Phase 2a part of the study is a single-arm evaluation of 1 dose level of Compound 1 (5 mg once daily [QD]) to establish proof of concept of Compound 1 in LR-MDS. Eligible subjects will receive Compound 1 for oral administration for the 16-week Core Period. Subjects who complete the 16-week Core Period will be eligible to continue receiving the same dose of Compound 1 for up to 156 weeks in the Extension Period. An overview of the Phase 2a study design is illustrated in FIG. 4.

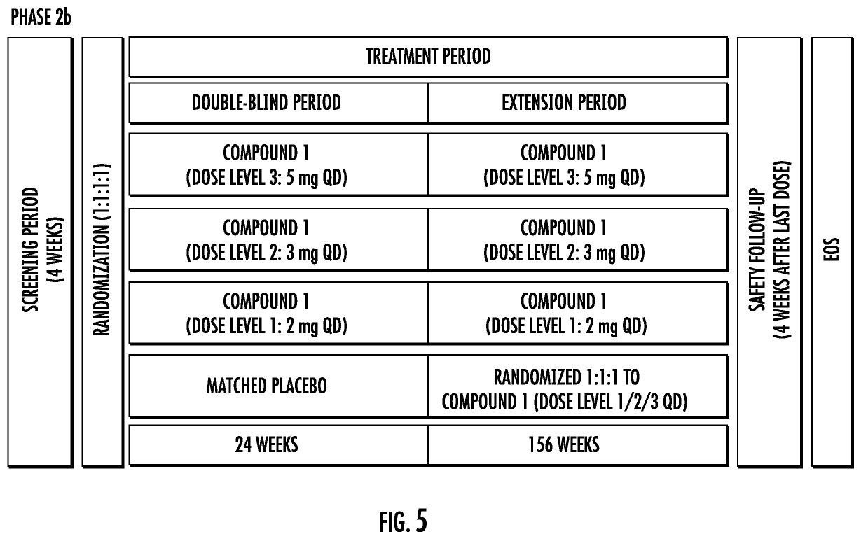

The Phase 2b part of the study is a double-blind, randomized, placebo-controlled evaluation of the efficacy and safety of Compound 1 (2 mg QD, 3 mg QD, and 5 mg QD) versus placebo. The initiation of the Phase 2b part of the study will be based on prespecified go/no-go criteria. Eligible subjects will be randomized in a 1:1:1:1 ratio to receive 2 mg QD Compound 1 (Dose Level 1), 3 mg Compound 1 (Dose Level 2), 5 mg QD Compound 1 (Dose Level 3) or matched placebo for QD oral administration. Subjects who complete the 24-week Double-blind Period will be eligible to receive Compound 1 for up to 156 weeks in the Extension Period. Randomization will be stratified by baseline transfusion burden (low transfusion burden [subjects who are NTD and subjects with LTB], high transfusion burden (HTB)). An overview of the Phase 2b study design is illustrated in FIG. 5.

During the Extension Period in Phase 2b, all subjects will receive Compound 1. Subjects who received placebo during the Double-blind Period will be randomized 1:1:1 to receive 2 mg QD Compound 1 (Dose Level 1), 3 mg QD Compound 1 (Dose Level 2), or 5 mg QD Compound 1 (Dose Level 3) to enable the assessment of the long-term safety and efficacy of these multiple doses of Compound 1. Subjects who received Compound 1 during the Double-blind Period will be eligible to continue receiving the same dose of Compound 1.

### Inclusion Criteria

Subjects are eligible to be included in the Phase 2a part of the study if all the following criteria apply:
1. At least 18 years of age at the time of providing informed consent.
2. Documented diagnosis of MDS according to World Health Organization (WHO) classification, that meets IPSS-R classification of lower-risk disease (risk score: <3.5) and <5% blasts as determined by the participant's bone marrow biopsy/aspirate during the Screening Period.
3. Nontransfused or with LTB, based on transfusion history from the participant's medical record, according to revised IWG 2018 criteria: a. NTD: <3 RBC units in the 16-week period before administration of the first dose of study drug and no transfusions in the 8-week period before administration of the first dose of study drug, or b. LTB: 3 to 7 RBC units in the 16-week period before administration of the first dose of study drug and <4 RBC units in the 8-week period before administration of the first dose of study drug.
4. An Hb concentration<11.0 g/dL during the 4-week Screening Period.
5. Eastern Cooperative Oncology Group (ECOG) Performance Status score of 0, 1, or 2.
6. If taking iron chelation therapy, the iron chelation therapy dose must have been stable and started≥56 days before administration of the first dose of study drug.
7. For women of childbearing potential (WOCBP) and men with partners who are WOCBP, must be abstinent of sexual activities that may result in pregnancy as part of their usual lifestyle or agree to use 2 forms of contraception, 1 of which must be considered highly effective, from the time of providing informed consent, throughout the study, and for 28 days after the last dose of study drug for women and 90 days after the last dose of study drug for men. The second form of contraception can be an acceptable barrier method.
8. Written informed consent from the participant before any study-related procedures are conducted and willing to comply with all study procedures for the duration of the study.

Subjects are eligible to be included in the Phase 2b part of the study if all the following criteria apply:
1. At least 18 years of age at the time of providing informed consent.
2. Documented diagnosis of MDS according to WHO classification that meets IPSS-R classification of lower-risk disease (risk score: ≤3.5) and ≤5% blasts as determined by the participant's bone marrow biopsy/aspirate during the Screening Period.
3. Nontransfused, with LTB, or with HTB, based on transfusion history from the participant's medical record, according to revised IWG 2018 criteria: a. NTD: ≤3 RBC units in the 16-week period before randomization and no transfusions in the 8-week period before randomization, or b. LTB: 3 to 7 RBC units in the 16-week period before randomization and ≤4 RBC units in the 8-week period before randomization, or c. HTB: ≥8 RBC units in the 16-week period before randomization and ≥4 RBC units in the 8-week period before randomization.
4. An Hb concentration≤11.0 g/dL during the 4-week Screening Period.
5. Up to 2 prior therapies including erythropoiesis-stimulating agents (ESAs) (e.g., erythropoietin [EPO], EPO+granulocyte colony-stimulating factor [G-CSF]) and/or luspatercept.
6. ECOG Performance Status score of 0, 1, or 2.
7. If taking iron chelation therapy, the iron chelation therapy dose must have been stable and started≥56 days before randomization.
8. For WOCBP and men with partners who are WOCBP, must be abstinent of sexual activities that may result in pregnancy as part of their usual lifestyle or agree to use 2 forms of contraception, 1 of which must be considered highly effective, from the time of providing informed consent, throughout the study, and for 28 days after the last dose of study drug for women and 90 days after the last dose of study drug for men. The second form of contraception can be an acceptable barrier method.
9. Written informed consent from the participant before any study-related procedures are conducted and willing to comply with all study procedures for the duration of the study

### Exclusion Criteria

Subjects are excluded from the Phase 2a part of the study if any of the following criteria apply:
1. Known history of acute myeloid leukemia (AML).
2. Secondary MDS, defined as MDS that is known to have arisen as a result of chemical injury or treatment with chemotherapy and/or radiation for other diseases.
3. Prior exposure to a pyruvate kinase activator, treatment administered for high-risk MDS (hypomethylating agents [HMAs], isocitrate dehydrogenase [IDH] inhibitors, or allogeneic or autologous stem cell transplant), and/or disease-modifying agents (e.g., immunomodulatory drugs such as lenalidomide). If a participant received≤1 week of treatment with a disease-modifying agent≥8 weeks before administration of the first dose of study drug, then they may not be excluded, at the Investigator's discretion.
4. Currently receiving treatment with luspatercept, EPO, or G-CSF. Treatment with EPO or G-CSF must have been stopped for ≥28 days before administration of the first dose of study drug; treatment with luspatercept must have been stopped for ≥65 days before administration of the first dose of study drug.
5. History of active and/or uncontrolled cardiac or pulmonary disease within 6 months before providing informed consent, including but not limited to:
   a. New York Heart Association Class III or IV heart failure or clinically significant dysrhythmia
   b. Myocardial infarction, unstable angina pectoris, or unstable hypertension; high risk thrombosis; hemorrhagic, embolic, or thrombotic stroke; deep venous thrombosis; or pulmonary or arterial embolism
   c. Heart rate-corrected QT interval using Fridericia's method of ≥470 milliseconds for female subjects and ≥450 milliseconds for male subjects, except for right or left bundle branch block
   d. Severe pulmonary fibrosis as defined by severe hypoxia, evidence of right-sided heart failure, and radiographic pulmonary fibrosis≥50%
   e. Severe pulmonary hypertension as defined by severe symptoms associated with hypoxia, right-sided heart failure, and oxygen indicated
6. History of hepatobiliary disorders, as defined by:
   a. Serum AST≥2.5×upper limit of normal (ULN) (unless due to hemolysis and/or hepatic iron deposition) and ALT≥2.5×ULN (unless due to hepatic iron deposition)
   b. Serum bilirubin>ULN, if the elevation is associated with clinically symptomatic choledocholithiasis, cholecystitis, biliary obstruction, or hepatocellular disease
7. Renal dysfunction, as defined by an estimated glomerular filtration rate (eGFR)≤45 mL/min.
8. Active infection requiring systemic antimicrobial therapy at the time of providing informed consent. If antimicrobial therapy is required during the Screening Period, screening procedures should not be performed while antimicrobial therapy is being administered, and the last dose of antimicrobial therapy must be administered≥7 days before administration of the first dose of study drug.
9. Major surgery within 12 weeks before administration of the first dose of study drug. Subjects must have completely recovered from any previous surgery before administration of the first dose of study drug.
10. History of any malignancy, except for nonmelanomatous skin cancer in situ, cervical carcinoma in situ, or breast carcinoma in situ. Subjects must not have active disease or have received anticancer treatment≤5 years before providing informed consent.
11. Positive test for hepatitis C virus (HCV) antibody (Ab) with evidence of active HCV infection, or positive test for hepatitis B surface antigen (HBsAg).
12. Positive test for HIV-1 Ab or HIV-2 Ab.
13. Absolute neutrophil count (ANC)≤500/L (0.5×109/L).
14. Platelet count≤75,000/L (75×109/L) assessed in the absence of platelet transfusions within 28 days before Screening.
15. Nonfasting triglyceride concentration≥500 mg/dL.
16. Receiving inhibitors of P-glycoprotein (P-gp) that have not been stopped for ≥5 days or a time frame equivalent to 5 half-lives (whichever is longer) before administration of the first dose of study drug.
17. Current enrollment or past participation (within 4 weeks or a time frame equivalent to 5 half-lives of the investigational study drug before administration of the first dose of study drug or, whichever is longer) in any other clinical study involving an investigational treatment or device.
18. Known allergy to Compound 1 or its excipients (silicified microcrystalline cellulose, croscarmellose sodium, sodium stearyl fumarate, and the Opadry® II Blue film coat [polyvinyl alcohol, titanium dioxide, macrogol/polyethylene glycol, talc, FD&C blue #2/indigo carmine aluminum lake/E132]).
19. Pregnant or breastfeeding.
20. Any medical, hematologic, psychological, or behavioral condition(s) or prior or current therapy that, in the opinion of the Investigator, may confer an unacceptable risk to participating in the study and/or could confound the interpretation of the study data.

Subjects are excluded from the Phase 2b part of the study if any of the following criteria apply:
1. Known history of AML.
2. Secondary MDS, defined as MDS that is known to have arisen as a result of chemical injury or treatment with chemotherapy and/or radiation for other diseases.
3. Prior exposure to a pyruvate kinase activator, including exposure to Compound 1 in the Phase 2a part of this study, treatment administered for high-risk MDS (HMAs, IDH inhibitors, or allogeneic or autologous stem cell transplant), and/or disease-modifying agents (eg, immunomodulatory drugs such as lenalidomide). If a participant received≤1 week of treatment with a disease-modifying agent≥8 weeks before randomization, then they may not be excluded, at the Investigator's discretion.
4. Currently receiving treatment with luspatercept, EPO, or G-CSF. Treatment with EPO or G-CSF must have been stopped for ≥28 days before administration of the first dose of study drug; treatment with luspatercept must have been stopped for ≥65 days before randomization.
5. History of active and/or uncontrolled cardiac or pulmonary disease within 6 months before providing informed consent, including but not limited to:
   a. New York Heart Association Class III or IV heart failure or clinically significant dysrhythmia
   b. Myocardial infarction, unstable angina pectoris, or unstable hypertension; high risk thrombosis; hemorrhagic, embolic, or thrombotic stroke; deep venous thrombosis; or pulmonary or arterial embolism
   c. Heart rate-corrected QT interval using Fridericia's method of ≥470 milliseconds for female subjects and ≥450 milliseconds for male subjects, except for right or left bundle branch block
   d. Severe pulmonary fibrosis as defined by severe hypoxia, evidence of right-sided heart failure, and radiographic pulmonary fibrosis≥50%
   e. Severe pulmonary hypertension as defined by severe symptoms associated with hypoxia, right-sided heart failure, and oxygen indicated
6. History of hepatobiliary disorders, as defined by:
   a. Serum AST≥2.5×ULN (unless due to hemolysis and/or hepatic iron deposition) and ALT≥2.5×ULN (unless due to hepatic iron deposition)
   b. Serum bilirubin>ULN, if the elevation is associated with clinically symptomatic choledocholithiasis, cholecystitis, biliary obstruction, or hepatocellular disease
7. Renal dysfunction, as defined by an eGFR≤45 mL/min
8. Active infection requiring systemic antimicrobial therapy at the time of providing informed consent. If antimicrobial therapy is required during the Screening Period, screening procedures should not be performed while antimicrobial therapy is being administered, and the last dose of antimicrobial therapy must be administered≥7 days before randomization.
9. Major surgery within 12 weeks before randomization. Subjects must have completely recovered from any previous surgery before randomization.
10. History of any malignancy, except for nonmelanomatous skin cancer in situ, cervical carcinoma in situ, or breast carcinoma in situ. Subjects must not have active disease or have received anticancer treatment≤5 years before providing informed consent.
11. Positive test for HCV Ab with evidence of active HCV infection, or positive test for HBsAg.
12. Positive test for HIV-1 Ab or HIV-2 Ab.
13. ANC≤500/L (0.5×109/L).
14. Platelet count≤50,000/L (50×109/L) assessed in the absence of platelet transfusions within 28 days before Screening.
15. Nonfasting triglyceride concentration≥500 mg/dL.
16. Receiving inhibitors of P-gp that have not been stopped for ≥5 days or a time frame equivalent to 5 half-lives (whichever is longer) before randomization.
17. Current enrollment or past participation (within 4 weeks or a time frame equivalent to 5 half-lives of the investigational study drug before randomization or, whichever is longer) in any other clinical study involving an investigational treatment or device.
18. Known allergy to Compound 1 or its excipients, including placebo (silicified microcrystalline cellulose, microcrystalline cellulose, croscarmellose sodium, mannitol, sodium stearyl fumarate, magnesium stearate, and the Opadry® II Blue film coat [polyvinyl alcohol, hypromellose, titanium dioxide, lactose monohydrate, macrogol/polyethylene glycol, triacetin, talc, FD&C blue #2/indigo carmine aluminum lake/E132]).
19. Pregnant or breastfeeding.
20. Any medical, hematologic, psychological, or behavioral condition(s) or prior or current therapy that, in the opinion of the Investigator, may confer an unacceptable risk to participating in the study and/or could confound the interpretation of the study data.

### Primary Endpoints Phase 2a

### Hemoglobin Response

Hemoglobin response is defined as a ≥1.5-g/dL increase from baseline in the average Hb concentration from Week 8 through Week 16. The proportion of subjects who achieve an Hb response (Hb response rate) will be summarized and the 2-sided 95% exact CI using the Clopper-Pearson method will be calculated. Hemoglobin concentrations assessed within 14 days after an RBC transfusion will be excluded from the analyses of the primary endpoint. Once this exclusion is applied, subjects who do not have any Hb concentration assessments from Week 8 through Week 16 will be considered non-responders.

### Transfusion Independence

Transfusion independence is defined as transfusion-free for ≥8 consecutive weeks during the Core Period (subjects with LTB only). The proportion of subjects who achieve TI (TI rate) will be summarized and the 2-sided 95% exact CI using the Clopper-Pearson method will be calculated.

### Secondary Endpoints Phase 2a

### Secondary Endpoints for Phase 2a Include:

AEs, SAEs, discontinuations due to AEs, and laboratory abnormalities during the Core Period.
Hb 1.0+response, defined as a ≥1.0-g/dL increase from baseline in the average Hb concentration from Week 8 through Week 16.
Change from baseline in Hb concentration during the Core Period.
≥1.5-g/dL increase from baseline in the Hb concentration at ≥2 consecutive time points from Week 8 through Week 16.
Change from baseline in total transfused red blood cell (RBC) units during the Core Period.
≥50% reduction in total transfused RBC units for ≥8 consecutive weeks during the Core Period compared with baseline.
Plasma concentration and pharmacokinetic parameters of Compound 1 during the Core Period.
Whole blood concentrations of pharmacodynamics parameters, including 2,3-diphosphoglycerate (2,3-DPG) and adenosine triphosphate (ATP) during the Core Period.

### Exploratory Endpoints Phase 2a

### Exploratory Endpoints for Phase 2a Include:

Change from baseline in exploratory biomarkers, including hepcidin, erythroferrone, soluble transferrin receptor, and growth differentiation factor 11 (GDF11) during the Core Period.
Change from baseline in iron, serum ferritin, total iron binding capacity, and transferrin saturation during the Core Period.
Change from baseline in markers of erythropoiesis, including absolute and percent reticulocytes, and erythropoietin during the Core Period.
Change from baseline in bone marrow-derived biomarkers, including erythroid precursors, during the Core Period.
Change from baseline in markers of hemolysis, including indirect bilirubin, lactate dehydrogenase, and haptoglobin during the Core Period.
Change from baseline in PKR activity during the Core Period.
Change from baseline during the Extension Period in:
   Total transfused RBC units (subjects with LTB only)
   Hb concentration
   Markers of iron metabolism
   Markers of erythropoiesis and hemolysis
   Bone marrow-derived biomarkers, including erythroid precursors
   Exploratory biomarkers
AEs, SAEs, discontinuations due to AEs, and laboratory abnormalities during the Extension Period.

### Primary Endpoints Phase 2b

The primary endpoint for Phase 2b is mHI-E response, defined as: 1)≥1.5-g/dL increase from baseline in Hb concentration for ≥8 consecutive weeks during the Double-blind Period (subjects who are NTD); 2) Transfusion independence, defined as transfusion-free for ≥8 consecutive weeks during the Double-blind Period (subjects with LTB only); and 3)≥50% reduction in total transfused RBC units for ≥8 consecutive weeks during the Double-blind Period compared with baseline (subjects with HTB only). Hemoglobin concentrations assessed within 14 days after an RBC transfusion will be excluded from the analyses of the primary endpoint. Once this exclusion is applied, subjects who are NTD and subjects with LTB will be considered as non-responders if the subjects do not have at least 2 Hb concentration assessments separated by≥8 weeks through Week 24.

### Secondary Endpoints Phase 2b

### Secondary Endpoints for Phase 2b Include:

AEs, SAEs, discontinuations due to AEs, and laboratory abnormalities during the Double-blind Period.
Change from baseline in Hb concentration during the Double-blind Period.
Change from baseline in total transfused RBC units from Week 8 through Week 24.
Transfusion independence, defined as transfusion-free for ≥8 consecutive weeks during the Double-blind Period.
Time to first mHI-E response during the Double-blind Period.
Maximum duration of mHI-E response for subjects who achieved an mHI-E response during the Double-blind Period.
Plasma concentration and pharmacokinetic parameters of Compound 1 during the Double-blind Period.
Whole blood concentrations of pharmacodynamics parameters, including 2,3-DPG and ATP during the Double-blind Period.
Exposure-response (or pharmacokinetic/pharmacodynamic) relationship between relevant pharmacokinetic parameters and endpoints that are indicators of clinical activity and safety during the Double-blind Period.

### Exploratory Endpoints Phase 2b

### Exploratory Endpoints for Phase 2b Include:

Change from baseline in the frequency of RBC transfusions during the Double-blind Period (subjects with LTB and HTB only)
Time to first transfusion during the Double-blind Period (subjects who are NTD only)
Change from baseline in exploratory biomarkers, including hepcidin, erythroferrone, soluble transferrin receptor, and GDF11 during the Double-blind Period
Change from baseline in iron, serum ferritin, total iron binding capacity, and transferrin saturation during the Double-blind Period
Change from baseline in markers of erythropoiesis, including absolute and percent reticulocytes, and erythropoietin during the Double-blind Period
Change from baseline in bone marrow-derived biomarkers, including erythroid precursors, during the Double-blind Period
Change from baseline in markers of hemolysis, including indirect bilirubin, lactate dehydrogenase, and haptoglobin during the Double-blind Period
Change from baseline in PKR activity during the Double-blind Period Change from baseline in the Anemia subscale of the Functional Assessment of Cancer Therapy questionnaire (FACT-An) during the Double-blind Period
Change from baseline in the Quality of Life in Myelodysplasia Scale (QUALMS) and the QUALMS Physical Burden (QUALMS-P) subscale during the Double-blind Period
Improvement in the Patient Global Impression of Severity (PGIS)-Symptoms of Anemia by at least 1 category during the Double-blind Period compared with baseline, or “no change” if no or mild symptoms of anemia at baseline
Improvement in the Patient Global Impression of Change (PGIC)-Symptoms of Anemia during the Double-blind Period, or “no change” if no or mild symptoms of anemia at baseline based on PGIS baseline score
Change from baseline in the Patient-Reported Outcomes Measurement Information System (PROMIS) Physical Function 4a questionnaire during the Double-blind Period
Change from baseline during the Extension Period in:
   Total transfused RBC units (subjects with LTB and HTB only)
   The frequency of RBC transfusions (subjects with LTB and HTB only)
   Hb concentration
   Markers of iron metabolism
   Markers of erythropoiesis and hemolysis
   Bone marrow-derived biomarkers, including erythroid precursors
   Exploratory biomarkers
AEs, SAEs, discontinuations due to AEs, and laboratory abnormalities during the Extension Period

### Dose Modifications

Excessive Hb response is defined as an increase in Hb concentration that is >ULN (by sex), in the absence of RBC transfusions. In the event of an excessive Hb response, in the absence of RBC transfusions for ≥4 weeks, study drug must be discontinued.

If a precipitous decrease in platelet count (e.g., ≥50% decrease from baseline) is observed, then the Investigator should monitor platelet count weekly and interrupt study drug for up to 28 days if clinically indicated. For the second occurrence or in the event of a Grade 4 platelet count decrease, discontinue study drug.

While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating anemia associated with myelodysplastic syndrome (MDS) in a subject suffering from MDS comprising administering to the subject a therapeutically effective amount of a compound having the structural formula: 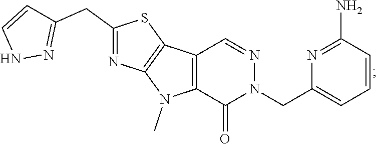 [SMILES: Cn1c2nc(Cc3cc[nH]n3)sc2c2cnn(Cc3cccc(N)n3)c(=O)c21]
or a pharmaceutically acceptable salt thereof.

2. A method of treating hemolytic anemia associated with myelodysplastic syndrome (MDS) in a subject suffering from MDS comprising administering to the subject a therapeutically effective amount of a compound having the structural formula: 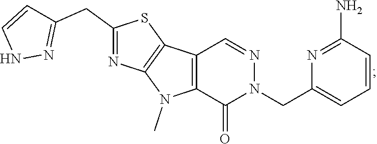 [SMILES: Cn1c2nc(Cc3cc[nH]n3)sc2c2cnn(Cc3cccc(N)n3)c(=O)c21]
or a pharmaceutically acceptable salt thereof.

3. A method of increasing the hemoglobin level in a subject suffering from myelodysplastic syndrome (MDS) comprising administering to the subject a therapeutically effective amount of a compound having the structural formula: 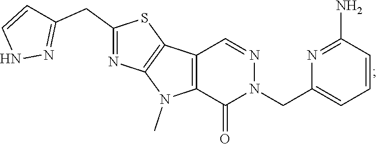 [SMILES: Cn1c2nc(Cc3cc[nH]n3)sc2c2cnn(Cc3cccc(N)n3)c(=O)c21]
or a pharmaceutically acceptable salt thereof.

4. A method of treating acquired PK deficiency (PKD) in a subject suffering from myelodysplastic syndrome (MDS) comprising administering to the subject a therapeutically effective amount of a compound having the structural formula: 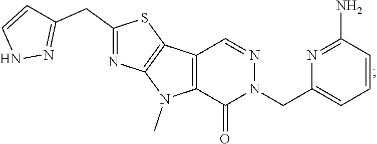 [SMILES: Cn1c2nc(Cc3cc[nH]n3)sc2c2cnn(Cc3cccc(N)n3)c(=O)c21]
or a pharmaceutically acceptable salt thereof.

5. A method of treating anemia associated with acquired PK deficiency (PKD) in a subject suffering from myelodysplastic syndrome (MDS) comprising administering to the subject a therapeutically effective amount of a compound having the structural formula: 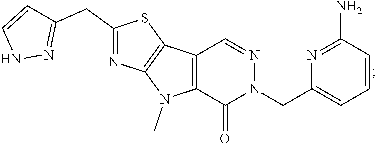 [SMILES: Cn1c2nc(Cc3cc[nH]n3)sc2c2cnn(Cc3cccc(N)n3)c(=O)c21]
or a pharmaceutically acceptable salt thereof.

6. A method of treating cytopenia in a subject suffering from myelodysplastic syndrome (MDS) comprising administering to the subject a therapeutically effective amount of a compound having the structural formula: 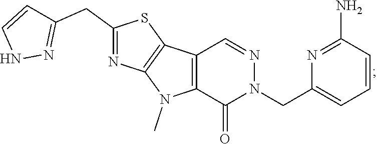 [SMILES: Cn1c2nc(Cc3cc[nH]n3)sc2c2cnn(Cc3cccc(N)n3)c(=O)c21]
or a pharmaceutically acceptable salt thereof.

7. The method of any one of claims 1 to 6, wherein the hemoglobin level of the subject improves over a period of at least 4 weeks, at least 6 weeks, at least 8 weeks, at least 10 weeks, at least 12 weeks, at least 14 weeks, at least 16 weeks, at least 18 weeks, or at least 20 weeks during treatment.

8. The method of any one of claims 1 to 6, wherein the hemoglobin level of the subject increases from baseline from week 1 through week 20, from week 1 through week 18, from week 1 through week 16, from week 4 through week 20, from week 4 through week 18, from week 4 through week 16, from week 6 through week 20, from week 6 through week 18, from week 6 through week 16, from week 8 through week 20, from week 8 through week 18, from week 8 through week 16, from week 10 through week 20, from week 10 through week 18, or from week 10 through week 16 during treatment.

9. The method of any one of claims 1 to 8, wherein the hemoglobin level of the subject increases from baseline from week 8 through week 16 during treatment.

10. The method of any one of claims 1 to 9, wherein the hemoglobin level of the subject increases from baseline at ≥2, ≥3, ≥4, ≥5, or ≥6 consecutive time points from week 1 through week 20, from week 1 through week 18, from week 1 through week 16, from week 4 through week 20, from week 4 through week 18, from week 4 through week 16, from week 6 through week 20, from week 6 through week 18, from week 6 through week 16, from week 8 through week 20, from week 8 through week 18, from week 8 through week 16, from week 10 through week 20, from week 10 through week 18, or from week 10 through week 16 during treatment.

11. The method of any one of claims 1 to 10, wherein the hemoglobin level of the subject increases from baseline at ≥2 consecutive time points from week 8 through week 16 during treatment.

12. The method of any one of claims 1 to 11, wherein the hemoglobin level of the subject increases from baseline by≥1.0 g/dL during treatment.

13. The method of any one of claims 1 to 12, wherein the hemoglobin level of the subject increases from baseline by≥1.5 g/dL during treatment.

14. The method of any one of claims 1 to 13, wherein the hemoglobin level of the subject increases from baseline by≥2.0 g/dL during treatment.

15. The method of any one of claims 1 to 14, wherein the subject becomes transfusion independent during treatment.

16. The method of any one of claims 1 to 15, wherein the subject is classified as having a low transfusion burden prior to treatment.

17. The method of any one of claims 1 to 16, wherein the subject becomes transfusion independent for ≥1 consecutive week, for ≥2 consecutive weeks, for ≥3 consecutive weeks, for ≥4 consecutive weeks, for ≥5 consecutive weeks, for ≥6 consecutive weeks, for ≥7 consecutive weeks, for ≥8 consecutive weeks, for ≥9 consecutive weeks, or for ≥10 consecutive weeks during treatment.

18. The method of any one of claims 1 to 17, wherein the subject becomes transfusion independent for ≥8 consecutive weeks during treatment.

19. The method of any one of claims 1 to 18, wherein the total transfused red blood cell (RBC) units of the subject are reduced from baseline by≥10%, ≥20%, ≥30%, ≥40%, ≥50%, ≥60%, ≥70%, or ≥80% during treatment.

20. The method of any one of claims 1 to 19, wherein the total transfused red blood cell (RBC) units of the subject are reduced from baseline by≥50% during treatment.

21. The method of any one of claims 1 to 20, wherein the total transfused red blood cell (RBC) units of the subject are reduced from baseline by≥50% over a period of ≥1 consecutive week, ≥2 consecutive weeks, ≥3 consecutive weeks, ≥4 consecutive weeks, ≥5 consecutive weeks, ≥6 consecutive weeks, ≥7 consecutive weeks, ≥8 consecutive weeks, ≥9 consecutive weeks, or ≥10 consecutive weeks during treatment.

22. The method of any one of claims 1 to 21, wherein the total transfused red blood cell (RBC) units of the subject are reduced from baseline by≥50% over a period of ≥8 consecutive weeks during treatment

23. The method of any one of claims 1 to 22, wherein the MDS is low risk MDS (as characterized by the Revised International Prognostic Scoring System (IPSS-R) for MDS).

24. The method of any one of claims 1 to 22, wherein the MDS is very low risk MDS (as characterized by the Revised International Prognostic Scoring System (IPSS-R) for MDS).

25. The method of any one of claims 1 to 22, wherein the MDS is intermediate risk MDS (as characterized by the Revised International Prognostic Scoring System (IPSS-R) for MDS).

26. The method of any one of claims 1 to 25, wherein the subject is male.

27. The method of any one of claims 1 to 25, wherein the subject is female.

28. The method of any one of claims 1 to 27, wherein the therapeutically effective amount of the compound administered to the subject is 2 mg daily, 3 mg daily, or 5 mg daily.

29. The method of any one of claims 1 to 28, wherein the therapeutically effective amount of the compound administered to the subject is 2 mg QD, 3 mg QD, or 5 mg QD.

30. The method of any one of claims 1 to 29, wherein the compound or pharmaceutically acceptable salt is administered orally.

31. The method of any one of claims 1 to 30, wherein the compound or pharmaceutically acceptable salt are in the form of a tablet or one or more granules.

32. The method of any one of claims 1 to 31, wherein the compound or pharmaceutically acceptable salt are in the form of one or more granules.

33. A method of treating anemia associated with low-risk myelodysplastic syndrome (MDS) in a subject suffering from MDS comprising orally administering to the subject 2 mg daily, 3 mg daily, or 5 mg daily of a compound having the structural formula: 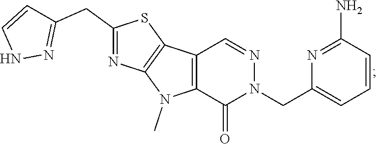 [SMILES: Cn1c2nc(Cc3cc[nH]n3)sc2c2cnn(Cc3cccc(N)n3)c(=O)c21]
or a pharmaceutically acceptable salt thereof in an amount a is equivalent to 2 mg daily, 3 mg daily, or 5 mg daily of the compound, wherein the subject is classified as being non-transfused, of low transfusion burden, or of high transfusion burden prior to administration.

34. The method of claim 33, wherein the subject is classified as having a low transfusion burden prior to treatment.

35. The method of claim 34, wherein the compound or pharmaceutically acceptable salt is administered to the subject for a period of 16 weeks.

36. The method of claim 34 or 35, wherein the subject becomes transfusion independent for ≥8 consecutive weeks during the 16 week administration period.

37. The method of any one of claims 34 to 36, wherein the hemoglobin level of the subject increases from baseline by≥1.0 g/dL, ≥1.5 g/dL or ≥2.0 g/dL from week 8 through week 16 of the 16 week administration period.

38. The method of claim 34, wherein the compound or pharmaceutically acceptable salt is administered to the subject for a period of 24 weeks.

39. The method of claim 38, wherein the subject becomes transfusion independent for ≥8 consecutive weeks during the 24 week administration period.

40. The method of claim 33, wherein the subject is classified as being non-transfused prior to treatment.

41. The method of claim 40, wherein the compound or pharmaceutically acceptable salt is administered to the subject for a period of 24 weeks.

42. The method of claim 41, wherein the hemoglobin level of the subject increases from baseline by≥1.0 g/dL, ≥1.5 g/dL or ≥2.0 g/dL for ≥8 consecutive weeks during the 24 week administration period.

43. The method of claim 33, wherein the subject is classified as having a high transfusion burden prior to treatment.

44. The method of claim 42, wherein the compound or pharmaceutically acceptable salt is administered to the subject for a period of 24 weeks.

45. The method of claim 43, wherein the total transfused red blood cell (RBC) units of the subject are reduced from baseline by≥50% over a period of ≥8 consecutive weeks during the 24 week administration period.

46. A method of treating a disease or disorder associated with mitochondrial dysfunction in a subject in need thereof comprising administering to the subject a therapeutically effective amount of a compound having the structural formula 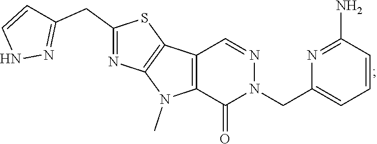 [SMILES: Cn1c2nc(Cc3cc[nH]n3)sc2c2cnn(Cc3cccc(N)n3)c(=O)c21]
or a pharmaceutically acceptable salt thereof.

47. A method of treating a disease or disorder associated with ineffective erythropoiesis in a subject in need thereof comprising administering to the subject a therapeutically effective amount of a compound having the structural formula 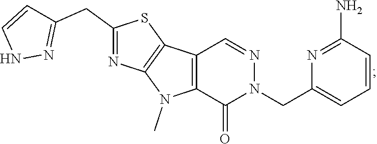 [SMILES: Cn1c2nc(Cc3cc[nH]n3)sc2c2cnn(Cc3cccc(N)n3)c(=O)c21]
or a pharmaceutically acceptable salt thereof.

48. The method of any one of claims 1 to 27, wherein the therapeutically effective amount of the compound administered to the subject is in the range of from 0.25 mg to 15 mg daily.

49. The method of any one of claims 1 to 27, wherein the therapeutically effective amount of the compound administered to the subject is in the range of from 0.25 mg to 2 mg QD or BID or from 1.5 mg to 5.5 mg QD or BID or from 4 mg to 6 mg QD or BID.

50. The method of any of claims 5 and 7 to 32 wherein the anemia associated with acquired PK deficiency (PKD) is hemolytic anemia.

51. The method of any one of claims 33 to 45 wherein the anemia associated with acquired PK deficiency (PKD) is hemolytic anemia.
